(19) United States
(12) Patent Application Publication
(10) Pub. No.: US 2009/0110601 A1
(43) Pub. Date: Apr. 30, 2009

(54) Molecularly imprinted polymers for detection in lateral flow devices

(76) Inventors: Raphael Levi, Netanya (IL); Ido Margalit, Gan Yavne (IL); Yarden Dloomy, Modiin (IL)

Correspondence Address: DR. D. GRAESER LTD., 9003 FLORIN WAY, UPPER MARLBORO, MD, 20772, US

(21) Appl. No.: 12/318,378

(22) Filed: Dec. 29, 2008

Related U.S. Application Data
(63) Continuation-in-part of application No. PCT/IL07/00789, filed on Jun. 28, 2007.
(60) Provisional application No. 60/806,783, filed on Jul. 9, 2006.
(60) Provisional application No. 61/016,829, filed on Dec. 27, 2007.
(60) Provisional application No. 61/027,462, filed on Feb. 10, 2008.

(51) Int. Cl. G01N 33/566

(52) U.S. Cl. 422/68.1

(57) ABSTRACT

Devices, methods and kits for rapid and simple determination of target molecules, including small molecules, polypeptides, proteins, cells and infectious disease agents in liquid samples that are capable of real-time measurement of these entities in fluid samples that are highly selective, highly sensitive, simple to operate, low cost, and portable. The devices, methods and kits also provide, in at least some embodiments, the use of MIPs in a flow through or lateral flow device.

This application is a continuation in part of PCT Application No. PCT/IL2007/00789 filed on Jul. 8, 2007 which claims priority from U.S. Provisional Application No. 60/806,783, filed on Jul. 9, 2006; and further claims priority from U.S. Provisional Application 61/016,829, filed Dec. 27, 2007, and U.S. Provisional Application 61/027,462, filed Feb. 10, 2008, all of which are hereby incorporated by reference as if fully set forth herein.

### FIELD OF THE INVENTION

The present invention relates to the field of diagnostics, and more particularly to flow through or lateral flow devices based on the use of molecularly imprinted polymers for measurement of analyte levels in a liquid sample, and methods and kits for use thereof.

### BACKGROUND OF THE INVENTION

Methods and devices for efficient and accurate detection and quantification of the level of analytes, such as protein related analytes, in liquid samples are of particular interest for use in a wide range of applications.

Currently, detection and measurement of protein molecules and protein containing organisms such as bacteria and viruses involves immunoassays that employ target specific antibodies. Rapid, real-time, and simple analysis of various analytes and protein related molecules of interest in liquid samples is performed today using sophisticated instruments and antibody based devices.

Antibodies are used in several areas, such as therapy, immunoaffinity purification and particularly in immunoassays. Antibodies are produced by immunization of animals with the respective antigen, leading to polyclonal antibodies, or by using fused cells, allowing the obtained cell lines to produce monoclonal antibodies.

Attempts to develop alternative methods of producing antibodies or antibody-like compounds involve recombinant techniques applied to bacteria or plants. Antibodies can be raised against most compounds and have been used in various applications (Kohler & Milstein, 1975, Nature 256, 495 497; Oellerich,. 1984, J. Clin. Chem. Clin. Biochem. 22, 895 904 Gosling, 1990. Clin. Chem. 36, 1408 1427; Kurstak, 1986, in Enzyme Immunodiagnosis, Kurstak, ed, pp 5 11, Academic Press, London), ranging from basic research to clinical analysis.

Immunological techniques using labeled reporters have been used widely in biological and medical research and in clinical diagnosis. Despite the abundance of known reporters and procedures, (Oellerich, 1984, Clin. Chem. Clin. Biochem. 22, 895-904; Gosling, 1990, Clin. Chem. 36, 1408-1427), all currently known immunodiagnostic techniques utilize the remarkable affinity and specificity of antibodies.

However, antibodies are labile bio-molecules, requiring careful handling and storage. Their production is a time-consuming procedure (Kurstak, 1986, in Enzyme Immunodiagnosis, Kurstak, ed, pp 5-11 Academic Press, London), including laborious steps such as conjugation of the hapten to a carrier protein, immunization of animals, bleeding of the animals and isolation and purification of the immunoglobulins.

Non-biological antibody mimics (artificial antibodies) are a useful alternative to natural antibodies. Use of such systems could reduce the need for animal sources. Furthermore, antibody mimics can be obtained against molecules against which it is generally considered to be difficult or impossible to raise antibodies, such as immuno-suppressive agents, short peptides or other small molecules. Such non-biological systems, prepared by chemical methods, would also be much more stable than natural antibodies, allowing repeated use, performance at higher temperatures, easy sterilization and increased batch to batch reproducibility.

An immunoassay-like technology in which stable and easily prepared highly selective reagents, such as synthetic polymers, rather than antibodies are used is known. This method utilizes molecular imprinted polymers (MIPs). A molecular imprint polymer is a polymer which is prepared by polymerizing functional monomers around a template or “print” molecule, which is then removed from the polymer by extraction or other means so that the polymer selectively absorbs the template or print molecule upon re-exposure to the print molecule. U.S. Pat. Nos. 5,821,311; 5,872,198; and 5,959,050 to Mosbach, et al. describe certain MIP polymers, a polymerization process, and symmetrical beads produced by suspension polymerization from functional monomers for use as chromatographic media

The method of molecular imprinting has attracted much attention in recent years (Alexander et al. 2006, J. Mol. Recognit.; 19: 106-180). Molecular imprinting originates from the concept of creating tailor-made recognition sites in polymers by template polymerization (Mosbach K. et al., Bio/Technology, 1996, 14, 163-170; Ansell R. J. et al., Curr. Opin. Biotechnol., 1996, 7, 89-94; Wulff G. Angew. Chem. Int. Ed. Engl., 1995, 34, 1812-32; Vidyasankar S. et al., Curr. Opin. Biotechnol., 1995, 6, 218-224; and Shea K. J, Trends In Polymer Science, 1994, 2, 166-173). Molecularly imprinted polymers have demonstrated remarkable recognition properties which have been applied in various fields such as drug separation (Fischer L., et al., J. Am. Chem. Soc., 1991, 113, 9358-9360; Kempe M, et al., J. Chromatogr., 1994, 664, 276-279; Nilsson K., et al., J. Chromatogr., 1994, 680, 57-61), receptor mimics (Ramstrom O., et al., Tetrahedron: Asymmetry, 1994, 5, 649-656; Ramstrom O., et al., J. Mol. Recogn., 1996, 9, 691-696; Andersson L. I., et al., Proc. Natl. Acad. Sci., 1995, 92, 4788-4792; Andersson L. I., Anal. Chem., 1996, 68, 111-117) bio-mimetic sensors (Kriz D., et al., Anal. Chem., 1995, 67, 2142-2144], antibody mimics (Vlatakis G., et al., Nature, 1993, 361, 645-647), template-assisted synthesis (Bystrom S. E., et al, J. Am. Chem. Soc., 1993, 115, 2081-2083) and catalysis (Muller R., et al., Makromol. Chem., 1993, 14, 637-641; Beach J. V., et al., J. Am. Chem. Soc., 1994, Vol. 116, 379-380).

Various methods for imprinting proteins and other macromolecules have been described. For example, ionic molecular images of polypeptides have been created by mixing a matrix material with the intact polypeptide chain to be bound by the molecular image (U.S. Pat. No. 5,756,717). Molecular imprints of cytochrome c, hemoglobin and myoglobin, respectively, have been prepared by polymerizing acrylamide in the presence of each intact protein (U.S. Pat. No. 5,814,223). An imprint of horse myoglobin selectively bound horse myoglobin from a mixture of proteins including whale myoglobin (U.S. Pat. No. 5,814,223).

A method for imprinting large biomolecules by the interfacial polymerization of a monomer in the presence of the print molecule and host polymer at the interface between an organic solvent and an aqueous solution is described in U.S. Pat. No. 6,582,971). Imprint compositions that comprise a matrix material defining an imprint of a template molecule, wherein the template molecule typically corresponds to a portion of a macromolecule of interest are described in U.S. Pat. No. 6,979,573).

It has been shown that MIPs can be prepared and targeted to Tobacco mosaic virus (Linden et al., 2006, Biomaterials 27, 4165-4168).

Molecular imprinting methods may involve “epitope imprinting” (Rachkov and Minoura, 2000, J. Chromatogr A.; 889, 111-118) wherein key epitopes are identified on the surface of the protein and the MIP is prepared with the linear peptide representing this epitope as target.

Numerous molecular imprinting-based analytical devices and methods for detection of various analytes have been reviewed by Ye and Haupt (Anal. Bioanal. Chem. 2004, 378, 1887-1897). A major challenge is to obtain an apparent signal from the polymer-analyte binding event. A variety of approaches have been proposed, yet the great majority of these involve sophisticated methods and machinery. Some examples of MIP-based sensors are as follows:

Yan et al. (U.S. Pat. No. 5,587,273) describe sensors employing molecularly imprinted film and measuring the capacitance or the light characteristics of the film after the exposing step or analyzing the film spectroscopically. MIP-based devices for detecting, analyzing and quantifying macromolecules are disclosed by Huang (U.S. Pat. No. 6,680,210). Detection is performed by dissociating the analyte molecules from the polymer after the binding and then analyzing them.

Williams et al. (U.S. Pat. No. 6,807,842) disclose a molecular recognition sensor system for detecting the presence and concentration of an analyte including a resistive sensor having a semiconductive polymer film which swells when exposed to the analyte.

Kroeger et al. (G.B. Pat. No. 2,337,332) disclose an electrode that has a surface modified with an imprinted synthetic polymer that specifically recognizes, binds and concentrates the analyte, in close proximity to the electrode surface. Either the bound analyte itself or an electrochemically active derivative pre-incubated with the electrode or added to the sample (competition/displacement assay) is quantified electrochemically (for example by differential pulse or square wave voltametry) directly at the electrode surface.

Penelle (U.S. Pat. No. 6,890,486) discloses an hexachlorobenzene MIP-based sensor by combining MIP techniques with quartz crystal microbalance (QCM).

Green et al. (U.S. Pat. No. 6,638,498) disclose devices utilizing MIPs with specific binding capacity for particular bile acids and/or salts, such as DCA and CDCA. The detection is performed by displacement of radioactive-labeled CDCA by CDCA in the sample. In a modification of the above, a fluorescent derivative of cholic acid is used as the assay substance.

Lawrence et al. (U.S. Pat. No. 6,833,274) disclose a cortisol fiber optic sensor using a cortisol-imprinted polymer and displacement of a cortisol-fluorescent chromophore conjugate.

Schwartz et al. (U.S. Pat. No. 6,967,103) disclose an explosive detector utilizing an array of MIP-coated, bifurcated fiber optic cables. Individual sensor fiber assemblies, each with a calibrated airflow, are used to expose the fibers to the target molecule. The detector energizes a dedicated excitation light source for each fiber and, through a detector comprising a filter and photodiode, simultaneously reads and processes the intensity of the resulting fluorescence that is indicative of the concentration of the target molecules.

Use of MIPs combined with displacement of analyte-marker conjugate has been shown to be practical in several laboratories (Vlatakis G. et al., Nature, 1993, 361, 645-647, Levi et al., 1997, Anal. Chem. 69. 2017-2021; Nathaniel T. et al., J. Am. Chem. Soc. 2005, 127, 5695-5700; Nicholls C. et al, Biosens. Bioelec., 2006, 21, 1171-1177). Competition of the analyte with the analyte-marker conjugate was also used with MIPs as a sensing method (Piletsky S. A. et al, Analytical Letters, 1997, 30, 445-455; Surugiu I. et al, Analyst, 2000, 125, 13-16).

PCT application WO 07/002,237 describes a method of analysing a liquid sample comprising contacting the sample with an MIP.

U.S. Pat. No. 6,890,486 describes use of quartz crystal microbalance sensors with MIP technology.

### SUMMARY OF THE INVENTION

The background art does not disclose the use of MIPs in a flow through or lateral flow device. Furthermore, the background art methods involve cumbersome sample preparation, and require use of sophisticated auxiliary analytical instrumentation by trained personnel.

There is thus a widely recognized need for, and it would be highly advantageous to have, rapid and simple devices and kits using MIPS for the diagnosis of small molecules, macromolecules, cells, and whole organisms, which are devoid of at least some of the limitations of the prior art.

The present invention, in various embodiments thereof, provides devices, methods and kits for rapid and simple determination of target molecules, including small molecules, polypeptides, proteins, cells and infectious disease agents in liquid samples that are capable of real-time measurement of these entities in fluid samples that are highly selective, highly sensitive, simple to operate, low cost, and portable. The devices, methods and kits also provide, in at least some embodiments, the use of MIPs in a flow through or lateral flow device.

The devices, methods and kits are suitable for use by untrained personnel without the need for uncommon and complicated to operate equipment. When the kits of the invention optionally require auxiliary equipment, it is preferably portable and hand held, relatively cheap, and simple to use.

The present invention provides a rapid and simple to use assay device, method and kit for determination of target polypeptides, proteins, cells and infectious disease agents in liquid samples, designed for use in the home, clinic, doctor's office, hospital bedside, factory or field. The assay devices achieve greater sensitivity than conventional rapid test assays, without compromising specificity, leading to stronger and/or more stable visual signals than those produced by conventional devices, easier interpretation of results, and reduced occurrence of indeterminate results. The devices may be used for detecting the target analytes in a variety of biological environmental and industrial samples in a short amount of time without the need for complicated sample preparation procedures, and thus are suitable for use by untrained personnel even in field conditions.

The present invention combines the advantages of the lateral and flow through devices (rapid, relatively cheap, and simple to use) with the advantages of MIPs for diagnostics (specific, controlled production, and very stable) with a novel double displacement/competition approach.

It is contemplated that the devices of the invention will, in at least some embodiments, provide a “real-time” measurement, i.e., a relatively rapid detection time, preferably of about 15 minutes more preferably less than 15 minutes. Alternatively, depending on the detectable reporter used, commercially available and simple to use auxiliary instrumentation such as readers, scanners, etc. may be used to interpret the results.

A target-specific molecular imprinted polymer (MIP) is used to detect the presence of the target analyte in a sample based on binding of the analyte by the MIP.

According to some embodiments of the present invention, there is provided a diagnostic device for detecting at least one analyte in a liquid sample, the device comprising a molecular imprinted polymer having analyte-specific binding sites fixed to a solid support, wherein the liquid sample is movable along a flow path along the solid support; a sample application area for applying the liquid sample to the device and bringing it in contact with the molecular imprinted polymer; and detection zone for detecting binding of said analyte to said molecular imprinted polymer.

According to some embodiments of the method or device of the present invention, the device comprises a lateral flow device, a flow through device, or a device combining lateral flow and flow through elements, wherein the liquid sample flows from the sample application area along or through the solid support components of the device along a flow path of the device.

According to some embodiments, the analyte displaces an analyte analog:reporter conjugate releasably bound to the molecular imprinted polymer, wherein displacement of the analyte analog:reporter conjugate indicates the presence of the analyte in the sample.

According to some embodiments, a releasable analyte analog:reporter conjugate is bound to the molecular imprinted polymer, wherein the affinity of the analyte for binding sites of the molecular imprinted polymer is at least equal to the affinity of the analyte analog:reporter conjugate for analyte-specific binding sites of the molecular imprinted polymer. Upon contacting the molecular imprinted polymer with the analyte in the liquid sample, the analyte is bound and the analyte analog:reporter conjugate is displaced. The device optionally further comprises a results zone comprising an analyte analog:reporter conjugate binding element immobilized to the solid support on the flow path of the sample, and the reporter conjugate binding element is capable of binding displaced analyte analog:reporter conjugate. Displacement of the analyte analog:reporter conjugate is optionally proportional to a concentration of the analyte in the liquid sample, such that detecting binding of the analyte to the molecular imprinted polymer comprises detecting binding of the displaced analyte:reporter conjugate to the analyte analog:reporter conjugate binding element.

According to some embodiments, the analyte displaces a first binding agent:analyte conjugate releasably bound to said molecular imprinted polymer, wherein the displaced first binding agent:analyte conjugate displaces a second binding agent:reporter conjugate releasably bound to the solid support, wherein displacement of the second binding agent:reporter conjugate indicates the presence of the analyte in the sample.

According to some embodiments, a releasable first binding agent:analyte conjugate is bound to the molecular imprinted polymer, wherein the affinity of the analyte for the binding sites of the molecular imprinted polymer is at least equal to the affinity of the first binding agent:analyte conjugate for the analyte-specific binding sites of the molecular imprinted polymer. Upon contacting the molecular imprinted polymer with the analyte in the liquid sample, the analyte is bound and the first binding agent:analyte conjugate is displaced. The device optionally further comprises a reporter-conjugate binding zone, comprising a reporter-conjugate binding element fixed to the solid support on the flow path of the liquid sample, the reporter-conjugate binding element having binding sites with a detectable, releasable, second binding agent:reporter conjugate attached thereto. Optionally and preferably, the affinity of the first binding agent:analyte conjugate for analyte-specific binding sites of the reporter conjugate binding element is at least equal to the affinity of the second binding agent:reporter conjugate for binding sites of the reporter:conjugate binding element. Binding of the first binding agent:analyte conjugate displaces the second binding agent:reporter conjugate, and displacement of the second binding agent:reporter conjugate is optionally and preferably proportional to a concentration of the analyte in the liquid sample. The device optionally further comprises a second binding agent:reporter conjugate binding element immobilized to the solid support on the flow path of the sample, the reporter conjugate binding element being capable of binding the displaced second binding agent:reporter conjugate. Detecting binding of the analyte to the molecular imprinted polymer optionally comprises detecting binding of the second binding agent:reporter conjugate to the analyte analog:reporter conjugate binding element.

According to some embodiments, the device of the present invention comprises an analyte analog:reporter conjugate, wherein the analyte competes with the analyte analog:reporter conjugate for analyte-specific binding sites of the molecular imprinted polymer, wherein the presence of unbound analyte analog:reporter conjugate is detected and indicates the presence of the analyte in the sample.

According to some embodiments, the device of the present invention comprises an analyte analog:reporter conjugate, wherein the affinity of the analyte for the analyte-specific binding sites of the molecular imprinted polymer is at least equal to the affinity of the analyte analog:reporter conjugate for analyte-specific binding sites of the molecular imprinted polymer. Upon contacting the molecular imprinted polymer with the analyte in the liquid sample, the analyte and analyte analog:reporter conjugate compete for binding sites of the molecular imprinted polymer. The device preferably further comprises a results zone comprising an analyte analog:reporter conjugate binding element bound to the solid support, the reporter conjugate binding element being capable of binding unbound analyte analog:reporter conjugate and providing a detectable signal that indicates the concentration of the analyte in the liquid sample.

According to some embodiments, the device of the present invention comprises a first binding agent:analyte conjugate, a second binding agent:reporter conjugate, and a second binding agent:receptor conjugate binding element. The analyte competes with the first binding agent:analyte conjugate competes for analyte-specific binding sites of the molecular imprinted polymer, such that in the presence of the analyte, at least a portion of said first binding agent:analyte conjugate is unbound. The unbound first binding agent:analyte conjugate then competes with the second binding agent:reporter conjugate for binding to the second binding agent:receptor conjugate binding element.

According to some embodiments, the device of the present invention comprises a first binding agent:analyte conjugate, wherein the affinity of the analyte for analyte-specific binding sites of the molecular imprinted polymer is at least equal to the affinity of the first binding agent:analyte conjugate for analyte-specific binding sites of the molecular imprinted polymer, wherein upon contacting said molecular imprinted polymer with the analyte in the liquid sample, the analyte and the first binding agent:analyte conjugate analyte compete for analyte-specific binding sites of the molecular imprinted polymer. Unbound first binding agent:analyte conjugate flows in a flow path of the liquid sample. The device optionally further comprises a second binding agent:reporter application area, comprising a second binding agent:reporter conjugate in a dry state; a reporter conjugate binding zone downstream, comprising a reporter-conjugate binding element fixed to the solid support on the flow path of the liquid sample. The affinity of the first binding agent:analyte conjugate to the reporter-conjugate binding element is at least equal to the affinity of the second binding agent:reporter conjugate to the binding sites. Upon contacting the dry second binding agent:reporter conjugate with the unbound first binding agent:analyte conjugate, the second binding agent:reporter conjugate and the unbound first binding agent:analyte conjugate compete for binding to the reporter-conjugate binding element, and unbound second binding agent:reporter conjugate flows downstream in the flow path of the liquid sample. The device optionally further comprises a results zone comprising a second binding agent:reporter conjugate binding element bound to the solid support, the reporter-conjugate binding element being capable of binding unbound second binding agent:reporter conjugate and providing a detectable signal that indicates the concentration of the analyte in the liquid sample.

According to some embodiments, the device of the present invention further comprises a first binding agent:analyte analog, wherein unbound first binding agent:analyte analog is produced by at least one of competition with the analyte for binding sites of the molecular imprinted polymer and displacement by said analyte from the molecular imprinted polymer. The device optionally further comprises a second binding agent:reporter conjugate binding element, wherein an unbound second binding agent:reporter conjugate is produced by at least one of competition with the first binding agent:analyte conjugate and displacement by the first binding agent:analyte conjugate, wherein the presence of unbound second binding agent:reporter conjugate indicates the presence of the analyte in the sample.

According to some embodiments, the first binding agent:analyte analog is provided either in the sample application area, or bound to the molecular imprinted polymer. The reporter conjugate binding element is optionally fixed to the solid support on the flow path of the liquid sample. The second binding agent:reporter conjugate is provided either in a reporter conjugate application area or releasably bound to the reporter conjugate binding element. The device preferably further comprises a results zone comprising a second binding agent:reporter conjugate binding element bound to the solid support, the reporter-conjugate binding element being capable of binding unbound second binding agent:reporter conjugate and providing a detectable signal that indicates the concentration of the analyte in the liquid sample.

According to some embodiments, the reporter-conjugate-binding element is immobilized horizontally to the sample liquid flow path on the solid support and unbound reporter conjugate saturates the binding sites of the reporter-conjugate binding element. The results are optionally visualized as an advancing column, having a length proportional to an amount of the analyte in the sample.

According to some embodiments, the device further comprising at least one of a positive control zone; a reference zone; an absorbent zone downstream of the results zone on the flow path of the liquid sample, the absorbent zone comprising absorbent material capable of absorbing the liquid sample; and a housing including at least one window aligned with at least one of the results zone, reference zone, and control zone to allow observation of test results on the device.

According to some embodiments, the intensity of a signal in the results zone is compared with the intensity of a signal in the reference zone, the device being arranged and constructed to provide a positive result when the analyte is present at or above a threshold level, to indicate the concentration of the analyte in the sample.

According to some embodiments, the device further comprises a reference zone, comprising at least one discrete band of a binding element comprising a known quantity of analyte analog:reporter conjugate, wherein the presence or absence of the target analyte in the sample is determined by comparison of the intensity of the signal from the reference zone with the intensity of the signal from the results zone.

According to some embodiments, the device is devoid of a reference zone, and the results zone further comprises a scale parallel to the immobilized reporter-conjugate binding element calibrated to correspond to the analyte concentration in the liquid sample being analyzed, such that the presence and concentration of analyte in the liquid is determined by the area covered by the reporter-conjugate along and through the reporter-conjugate binding element in the results zone.

According to some embodiments, the analyte is selected from the group consisting of a cell, an organism, a small molecule, a protein, a hormone, an enzyme, a biomarker, metabolites of biomarkers, metabolites of drugs, a drug, a drug metabolite a drug-protein conjugate, a drug metabolite-protein conjugate, a vitamin, a drug of abuse, a natural or synthetic toxin, a chemical or biological warfare agent, antibodies to a drug, antibodies to infectious agents, an environmental pollutant, an immunoglobulin, a lymphokine, a cytokine, a soluble cancer antigen, a growth factor, a neurotransmitter, a molecule indicating the safety or quality of a foodstuff, a process chemical, a byproduct of a production process, a pesticide, an insecticide, a herbicide, a fertilizer, a surfactant, an adhesive, and an agent used in the manufacture of food, industrial agents or chemical products.

According to some embodiments, there is provided a kit comprising the device of the present invention; optionally, one or more reagents or compositions for extracting or processing the sample to elute the analyte; optionally, one or more diluents; and optionally, instructions for practicing a method of detecting and determining the presence, absence or concentration of a target analyte in a liquid sample.

The foregoing and other features and advantages of the invention will be apparent from the following description which proceeds with reference to the accompanying Figures and claims.

### BRIEF DESCRIPTION OF THE DRAWINGS

The invention is herein described, by way of example only, with reference to the accompanying drawings and the following detailed description, it being understood that the particulars shown are by way of example and illustrative discussion only, and are presented to provide what is believed to be the most useful and readily understood description of the embodiments of the invention. No attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a side view illustration of a first embodiment of a lateral flow device with competition or single displacement and vertical visualization as described in Example 1.

FIG. 1B is a side view illustration of lateral flow device with competition or single displacement and horizontal visualization as described in Example 2.

FIG. 2A is a side view illustration of lateral flow device with competition/double displacement and vertical visualization as described in Example 3.

FIG. 2B is a side view illustration of lateral flow device with competition/double displacement and horizontal visualization as described in Example 4.

FIG. 3A is a side view illustration of flow through device with competition or single displacement and vertical visualization as described in Example 5.

FIG. 3B is a side view illustration of flow through device with competition or single displacement and horizontal visualization as described in Example 6.

FIG. 4A is a side view illustration of flow through device with competition/double displacement and vertical visualization as described in Example 7.

FIG. 4B is a side view illustration of flow through device with competition/double displacement and horizontal visualization as described in Example 8; and

FIG. 5 is a side view illustration of a lateral flow as described in Example 9

### DESCRIPTION OF THE PREFERRED EMBODIMENTS

The present invention provides diagnostic methods, devices, and kits for determining the presence or absence or concentration of at least one analyte in a liquid sample.

The diagnostic methods, devices and kits according to the present invention may be better understood with reference to the accompanying figures, examples, and description. It is contemplated that the invention is not limited in its application to the details set forth in the following description or drawings, or exemplified by the Examples. The invention may be practiced in various other ways and is capable of other embodiments. Also, it is contemplated that the phraseology and terminology used herein are for purposes of description and should not be regarded as limiting.

Before describing particular methods and devices of the invention, the general features of the invention for at least some embodiments will be described.

According to some embodiments, there is provided a diagnostic device for detecting at least one analyte in a liquid sample, the device comprising at least one solid support, to which is bound a molecular imprinted polymer (MIP) having analyte-specific binding sites; a sample application area; and a detection zone.

The MIP is bound to the solid support either by physical means or by chemical bonding (immobilization) of the object to the support.

The liquid sample is moveable along a flow path along or through the solid support, such as, for example by capillary action or by any type of flow.

The liquid sample is applied to the sample application area, and brought into contact with the molecular imprinted polymer, which is downstream of the sample application area, on the flow path of the liquid sample.

Binding of the analyte to the molecular imprinted polymer is determined in the detection zone, which is preferably situated downstream of the location of the molecular imprinted polymer. Preferably, the detection zone provides a detectable signal that indicates the presence of the analyte in the liquid sample. However, it should be noted that optionally such detection involves the direct detection of the analyte in the sample, while alternatively (or additionally), such detection optionally involves indirect detection of the analyte in the sample, for example according to competition and/or displacement, or a combination thereof.

According to some embodiments, there is provided a method of detecting at least one analyte in a liquid sample, the method comprising providing a diagnostic device for detecting at least one analyte in a liquid sample, the device comprising at least one solid support, to which is bound a molecular imprinted polymer having analyte-specific binding sites, a sample application area, and a detection zone; applying the liquid sample to the sample application area and detecting binding of the analyte to the molecular imprinted polymer.

As used herein, the term ‘analyte’ refers to a molecule, group of molecules or compound of natural or synthetic origin sought to be detected or measured, or an analogue or derivative thereof, that is capable of binding specifically to at least one binding partner (e.g., MIP). Analogues or derivatives may be used when they participate in an assay, as one member of a binding pair, in a manner that is substantially equivalent to that of the analyte itself.

The methods of this invention may be practiced with assays for virtually any analyte. Analytes vary in size and may range from small molecules, polypeptide analytes, having less than 30 amino acids, to whole organisms, such as bacteria.

The target analyte may be any molecule, polypeptide, protein or infectious disease agent of interest. Any known analyte to which an appropriate analyte-specific MIP may be prepared may be easily detected and/or quantified using the disclosed methods and devices. The methods and devices described herein can be used in the context of basic scientific research, the practice of medicine, including veterinary and dental medicine, forensic analysis, environmental protection monitoring and studies, industrial or chemical manufacturing, and the development and testing of pharmaceutical, food, and cosmetic products.

The analytes detected may include, but are not limited to, analytes selected from the group consisting of a bio-marker, pesticide, drug of abuse protein, a hormone, an enzyme, a biomarker, a natural or synthetic toxin, chemical warfare agent, biological warfare agent, antibodies to a drug, antibodies to infectious agents, an immunoglobulin, a lymphokine, a cytokine, a soluble cancer antigen, a growth factor and an infectious disease agent.

As examples, but not limiting in any way, the analyte may comprise a biomarker (both for humans and for animals) selected from the group consisting of methylmalonic acid (MMA), homocysteine, creatinine, TSH, BNP, FSH, troponin, CK, CKMB, AST, GOT, LDH, Myoglobin, PSA, AST, ALT, ALKP, GGT and Bilirubin. The analyte may comprise an infectious disease agent selected from the group consisting of Avian influenza, HIV virus, Hepatitis virus, Polio virus, Cytomegalovirus, Dengue, Ebola, Herpes virus, Rubeola, EBV, Rabies virus, Respiratory Syncytial Virus, Rotavirus, SARS virus, West Nile virus, Yellow fever virus, Campylobacter, Chlamydia, Cholera, Clostridium, Diphtheria, E. Coli, Neisseria, Helicobacter, Haemophilus, Mycobacterium, Staphylococcus, Pertussis, Salmonella, Shigella, Streptococcus, Treponema, Tetanus and Yershinia. The analyte may comprise a toxin selected from the group consisting of staphylococcal enterotoxin, staphylococcal enterotoxin B, ricin, botulinum toxin, mycotoxin, tetanus toxin, cholera toxin and trichothecene mycotoxins. The analyte may be selected from the group consisting of antibodies specific to infectious disease agents or auto-antibodies related to auto-immune diseases.

According to some embodiments, the liquid sample of the present invention optionally comprises any liquid that might contain a specific target analyte. Such liquids include biological fluids, environment samples, foodstuffs, drugs, toxins, industrial samples and byproducts of industrial production procedures. The biological fluid is optionally and preferably selected from the group consisting of body fluids, liquid obtained from breath, tissue homogenates, and process fluids.

The liquid sample may optionally comprise an environmental sample selected from the group consisting of liquids, such as water, oil, liquid waste, or liquid extracted from solids, such as liquid extracted from solid waste, soil and plants.

The sample can optionally be used as obtained directly from the source or following a pretreatment so as to modify its character. Pretreatment may involve, for example, separating plasma from blood, diluting viscous fluids, diluting the sample or the like. Methods of treatment can involve filtration, distillation, concentration, inactivation of interfering components, and the addition of reagents. For example, the sample may be centrifuged or filtered to remove particulate matter, or may be dissolved in or supplemented by a buffer or surfactants to provide a suitable medium in order to allow more efficient detection of the analyte. Suitable buffers include any of those known to skilled artisans, such as a 1-1,000 mM solution of Tris (TRIZMA, Sigma Chemical Co., St. Louis, Mo.), or 1-1,000 mM TRIS (2-Amino-2-(hydroxy-methyl)-1,3-propanediol) or 0.05-0.5% of the surfactant Polysorbate 20 (commercially also known as Tween® 20). Other buffers include phosphate buffered saline (PBS), citrate buffer, or bicarbonate buffer.

The present invention may also optionally be used to detect analytes that are initially contained within solid-phase samples. These analytes would simply be extracted and suspended or dissolved in liquid prior to analysis using appropriate reagents or compositions for extracting or processing the sample to elute the analyte. The extraction process could be as simple as shaking a solid sample in a diluent or buffer such as those listed above, which could then be applied to the solid support. Optionally, equipment such as a manual press, mortar and pestle, homogenizer, juicer, mixer or food processor may be used to pre-treat or extract a sample to bring the target analyte into liquid form suitable for testing in the device. Solid samples may include biological tissues (obtained, for example, in the process of performing a biopsy), soil, or foliage.

According to some embodiments, the analyte-specific molecular imprinted polymer (MIP) may be prepared in accordance with any technique known to those skilled in the art. These methods include covalent imprinting (Wullf G, 1982, Pure & Appl. Chem., 54, 2093-2102) whereby the monomers are covalently attached to the analyte and polymerized using a cross-linker. Subsequently, the analyte is cleaved from the polymer leaving analyte-specific binding cavities. Alternatively, a non-covalent imprinting method such as disclosed by Mosbach (U.S. Pat. No. 5,110,833) may be used, whereby the monomers interact with the target molecule by non-covalent forces and are then connected via a cross-linker to form target specific binding sites after removal of the target molecule. Variations on these methods may be used to construct thin molecularly imprinted films and membranes (Hong et al. 1998 Chem. Mater., 10, 1029-1033); imprinting on the surface of solid supports (Blanco-López, et al, 2004, Anal. Bioanal. Chem., 378, 1922-1928; Sulitzky C. et al, 2002 Macromolecules, 35, 79-91); microspheres (Ye et al., 2000, Macromolecules, 33, 8239-8245) and even proteins and whole microorganisms (Hayden et al 2006 Adv. Funct. Mater., 16, 1269-1278)

Preferably, the molecular imprint polymer comprises a polymer polymerized from monomers cross-linked with cross-linker in the presence of the target analyte and a porogen, the polymer having a capacity for selectively binding the target analyte. A relevant method for proteins imprinting is the so called “epitope imprinting” (Rachkov and Minoura, 2000, J. Chromatogr A.; 889, 111-118) where key epitopes are identified on the surface of the protein and the MIP is prepared with the linear peptide representing this epitope as target.

The MIP may be prepared against the analyte or to any conjugate of the analyte with other molecules, providing that the MIP binds specifically both the analyte and its conjugate.

According to some embodiments of the device of the present invention, the MIP is provided in an MIP-conjugate zone, which is a zone on the device comprising analyte-specific molecular imprinted polymer (“MIP”) possessing specific binding affinity for the tested analyte, either to the whole analyte, to a discrete portion of the analyte or to a peptide representing a specific epitope on the analyte, fixed to the solid support, either directly or indirectly.

According to some embodiments, the analyte-specific binding sites of the MIP are saturated with molecules of either a releasable analyte analog:reporter conjugate, or a releasable first binding agent:analyte conjugate.

As used herein the term ‘analyte specific’ refers to a binding reaction which is determinative of the presence of the analyte in the presence of a heterogeneous population of molecules.

As used herein, the term ‘analyte analog’ refers to a modified analyte or any other molecule or combination thereof, that has structural similarity to the form of the analyte as potentially found in the sample, and which can bind to at least one analyte binding partner. In certain embodiments of the invention, the analyte analog is a detectable analyte analog-reporter conjugate. For instance, an analyte analog-conjugate may comprise the analyte conjugated to biotin or to any other reporter pair binding element, in which biotin is an example of such an element, forming one-half of the reporter pair. The analyte analog can be MMA, a target protein or a peptide representing a discrete epitope on the tested analyte.

As used herein, the term ‘reporter’ refers to any molecule, particle or composition that is capable of being attached to an analyte, analyte analog or binding partner that is detectable by spectroscopic, photochemical, biochemical, immunochemical, electrical, optical, radio-activity detection or chemical means. The reporter is selected from a large variety of available molecules and particles, according to the desired attributes of the analytical device. Some examples of such reporters, which are not to be considered as limiting in any way, include: HABA, dyes, fluorescent dyes, radiolabels, magnetic particles, metallic particles, colored particles (such as gold and latex sols), fluorescent particles, metal sols, enzyme substrates and enzymes, chemiluminescent molecules, photosensitizers, semiconductor particles, quantum dots and suspendable particles. The coupling of a compound (e.g., an analyte) to a reporter can be through covalent bonds, ionic bonds, hydrogen bonding, chelate formation, adsorption processes, hydrophobic interactions, hydrophylic interactions, electrostatic interactions and the like, or any combinations of these bonds and interactions and/or may involve a linking group.

The detectable reporter is optionally selected from the group consisting of HABA, dyes, fluorescers, fluorescent dyes, radiolabels, magnetic particles, metallic particles, colored particles, metal sols, enzyme substrates, enzymes, chemiluminescers, photosensitizers, suspendable particles, polymer particles, semiconductor particles, quantum dots.

As evident from the preceding list, the detectable reporter may be a visible substance, such as a colored latex bead, or it may participate in a reaction by which a colored product is produced. The reaction product may be visible when viewed with the naked eye, or may be apparent, for example, when exposed to a specialized light source, such as ultraviolet light. Although it is expected that viewing the result zone (either directly or indirectly) will be the primary way in which the test result is obtained, other methods, for example where the analyte is associated with a fluorescent substance that is detected by subsequent exposure to a scanner, are also considered within the scope of the invention.

The concentration of analyte in the sample, whether qualitative or quantitative, is indicated by the amount of the detectable reporter which subsequently becomes associated with the results zone. A detectable signal that indicates the concentration of the analyte is produced in the results zone.

As used herein, the term ‘analyte analog:reporter conjugate’ refers to a conjugate used for detection of small molecules and when the “epitope imprinting” method is used in the production of the analyte specific MIP. The synthetic peptide, or other synthetic or naturally obtained molecule, which may represent a single molecule or group of such molecules, represents the analyte specific epitope or its analog and is conjugated to a reporter molecule, which may for example be selected from the group of molecules discussed below, such as gold sol. The analyte analog:reporter conjugate has high binding affinity to the analyte-specific MIP, but lower than the affinity of the unmodified analyte. The conjugated molecules remain attached to the binding cavities of the analyte-specific MIP even in a moist state unless they are displaced, optionally and preferably in a dose-dependent manner, by the target analyte in the tested liquid sample. When displaced from the polymer the analyte analog:reporter conjugate is freely mobile within the moist solid support.

As used herein, the term ‘analyte analog:reporter:binding partner conjugate’ refers to a conjugate used when imprinting the whole polypeptide/protein or other molecule or group of molecules. The target analyte or its analog conjugated to a reporter from the group of molecules and particles discussed below, such as gold sol, as well as to a binding partner, from the group discussed bellow, such as biotin. The analyte analog:reporter:binding partner conjugate has high binding affinity to the analyte-specific MIP, but lower than the affinity of the unmodified analyte. The conjugated molecules remain attached to the binding cavities of the analyte-specific MIP even in a moist state unless they are displaced, in a dose-dependent manner, by the target analyte in the tested liquid sample. When displaced from the polymer the analyte analog:reporter:binding partner conjugate is freely mobile within the moist solid support. In the case of polypeptides and proteins, the analyte analog:reporter:binding partner conjugate is conjugated simultaneously to reporter such as gold sol as well as to binding agents, such as biotin, that can be used to capture the displaced analyte analog:reporter:binding partner conjugate.

As used herein, the term ‘binding affinity’ refers to the strength of binding of one molecule to another. If a particular molecule binds to or specifically associates with another particular molecule, these two molecules are said to exhibit binding affinity for each other. Binding affinity is related to the association constant and dissociation constant for a pair of molecules, but it is not critical to the invention that these constants be measured or determined. Rather, affinities as used herein to describe interactions between molecules of the described methods and devices are generally apparent affinities (unless otherwise specified) observed in empirical studies, which can be used to compare the relative strength with which one molecule (e.g., a MIP or other specific binding partner) will bind two other molecules (e.g., an analyte and an analyte-reporter conjugate). The concepts of binding affinity, association constant, and dissociation constant are well known.

According to some embodiments, the analyte-specific binding sites of the MIP are unoccupied, and are available for binding by either the analyte or by a binding competitor.

According to some embodiments, the MIP is TSH-specific. Optionally, such a MIP comprises a polymer polymerized from Methacrylic acid (MAA) as monomer, ethylene glycol dimethacrylate (EGDMA) as cross linker, 2,2P-azobis(2,4-dimethylvaleronitrile) (ABDV) as initiator and 3% water in acetonitrile as porogen, in the presence of the synthetic peptide corresponding to an epitope on the TSH β subunit, such as the amino acid residues at positions 95-112 of the TSH β subunit (LSCKCGKCNTDY). The synthetic peptide may optionally be prepared on a conventional peptide synthesizer using standard procedures.

According to some embodiments of the device of the present invention, the solid support may comprise any porous material used in the flow devices industry and having a porous structure that facilitates liquid advancement along the device by capillary forces. Preferably, the solid support is selected from the group consisting of a porous material, a porous membrane, a granular material, and an absorbent material. These materials often possess also good binding capabilities for proteins. Examples to of such materials, which are not limiting in any way, include: Nitrocellulose (High Protein Binding) Cellulose Acetate (Low Protein Binding) Glass Fiber Membranes (Non-Protein Binding), membranes made of Nylon, Polyvinylidene Fluoride (PVDF), poly Ether Sulfone (PES) or other membranes of this nature that are available or will be developed by companies specializing in diagnostic membranes manufacturing, such as Millipore Corporation, Pall Corporation and Whatman as well as novel micro-fluidic systems, such as the system disclosed in U.S. Pat. Application No. 20050042766.

The devices may optionally comprise more than one solid support, whereby more than one analyte can be detected in a sample.

According to some embodiments of the device of the present invention, the sample application area comprises an absorbent pad made of bibulous, porous or fibrous material capable of absorbing liquid rapidly. Its porosity may be unidirectional (i.e., with pores or fibers running wholly or predominantly parallel to an axis of the flow) or multidirectional (i.e., omnidirectional, a sponge-like structure). A porous plastic material, such as polypropylene, polyethylene (preferably of very high molecular weight), polyvinylidene flouride, ethylene vinylacetate, acrylonitrile and polytetrafluoro-ethylene may be used. Pre-treatment with a surface-active agent during manufacture may optionally be used to reduce any inherent hydrophobicity and improve the ability to take up and transport a moist sample rapidly and efficiently. The material may also be made of paper or other cellulosic materials. The material connects an opening in the casing of the device with the solid support of the device.

A pad separating serum from whole blood, such as described in U.S. Pat. No. 5,660,798, may be used with devices that test analytes in serum. A sample applied to the sample application area is absorbed by the absorbent pad, flows freely by capillary forces and comes in contact with the solid support. Depending on the type of device, the volume of the sample that enters the device may be either random or controlled by a smart application pad that allows a precise pre-determined sample volume to enter the device (see for example U.S. Pat. No. 6,008,056).

According to some embodiments, the device of the present invention further comprises a casing. The casing keeps the solid support in a suitable configuration in order to ensure correct functioning of the entire device.

The casing optionally comprises windows, preferably between 1 and 4 windows. More preferably, the casing comprises a results window that serves to allow observation of the result. Further preferably, the casing comprises a control window, which allows for viewing of a control reaction, e.g., to confirm adequate performance of the test. Also optionally, the casing comprises a third window or opening in the casing allows application of the liquid sample to the sample application area, at the sample window, either by direct placement of the device in the sample (allowing contact of the sample at the open window), or by application of the sample with a dropper or a similar device to the sample window. Additionally, the casing may optionally comprise a reference window to allow comparison of the intensity of the results in the results zone with the intensity of indicator bands in the reference zone.

Optionally, the solid support further comprises a reference zone. As used herein, the reference zone is a zone where several lines of analyte analog-reporter binding element are immobilized to the solid support, having known amounts of the analyte analog-reporter conjugate bound to them and producing a distinctive signal with intensity proportional to the amount of the bound reporter.

The device may optionally further comprise a positive control zone comprising means for generating a positive control confirming the proper flow and binding of the analyte analog:reporter conjugate to the results zone to thereby determine that a test is working.

Optionally, the devices of the invention may include one or more absorbent pads that are positioned to facilitate the flow of the analyte through the results zone, the reference zone, and/or the control zone.

Additionally, the support may optionally include an absorbent zone comprising a pad of absorbent material in fluid communication with the solid support when the pad and solid support are wet, the pad having sufficient porosity and capacity to absorb excess liquid.

According to some embodiments, the device of the present invention may comprise either a lateral flow device, which is intended to include devices with a dipstick format, or a flow-through device, or a combination thereof.

According to some embodiments, the detection system may be either by single displacement or competition and direct monitoring of the conjugated reporter or by the double displacement or competition approach, as will be explained in further detail below. However, for any of the below embodiments, optionally flow through or lateral flow may be implemented.

### Single Displacement

According to some embodiments of the present invention, there is provided a single displacement diagnostic device for determining the presence, absence or concentration of a target analyte present in a liquid sample. The device comprises an analyte analog:reporter conjugate releasably bound to a molecular imprinted polymer on a solid support; a sample application area for applying the sample to the device and bringing it in contact with the solid support; and a detection area. The analyte has an affinity for the binding sites of the MIP which is equal or greater than that of the analyte analog:reporter conjugate, such that contact between the analyte and the MIP causes the analyte analog:reporter to be displaced.

In a further aspect, the invention is directed to a method for detecting and determining the presence, absence or concentration of a target analyte present in a liquid sample by an assay comprising the steps of applying a sample suspected of containing the analyte to the sample application area of the single displacement diagnostic device of the invention and allowing the sample to flow along or through the solid support and contact the MIP-conjugate zone so that, if analyte is present in the sample, analyte binds to the binding sites of the MIP, displacing analyte analog:reporter conjugate which flows to the results zone where it is captured by the analyte analog:reporter conjugate binding element, producing a detectable signal that indicates the presence or amount of the analyte in the sample.

The detection area preferably comprises a results zone comprising an analyte analog:reporter conjugate binding element immobilized to the solid support, wherein the reporter conjugate binding element is capable of binding the displaced analyte analog:reporter conjugate.

As used herein, the term ‘analyte analog:reporter conjugate binding element’ refers to a zone on the device comprising element(s) that possess high affinity to the analyte analog:reporter conjugate molecule. These elements may optionally comprise, but are not limited to, biotin binding elements (for example Avidin, StrepAvidin, NeutrAvidin and ExtrAvidin), reporter-specific antibodies, enzymes, substrates and MIP prepared specifically against the analyte analog:reporter conjugate.

The single displacement method comprises introducing a liquid sample which is suspected of containing the analyte to be tested for, into the sample application area, and permitting the sample to migrate along the support. If the analyte is present in the sample, it binds to the analyte-specific binding sites of the MIP, and displaces the analyte analog:reporter conjugate. The displaced reporter conjugate migrates along or through the solid support to the results zone, where it binds to the reporter conjugate binding element, providing a detectable signal. The presence and/or intensity of the detectable signal is preferably at least related to the amount of analyte in the sample, whether quantitatively or qualitatively, although optionally a binary “yes/no” answer may be obtained; this is also optionally possible as for the other embodiments below.

### Double Displacement

In another aspect, the invention is directed to a double displacement diagnostic device for directly detecting and determining the presence, absence or concentration of a target analyte in a liquid sample. The device comprises a solid support to which is bound an MIP, and a releasable first binding agent:analyte conjugate bound to the MIP; a sample application area; and a reporter:conjugate binding zone downstream of the sample application area.

The double displacement approach is designed to facilitate signal formation and interpretation and that may be applied in other apparatus of similar nature. This approach finds particular utilization with the well-established biotin-avidin system that offers very strong binding affinity together with a multitude of analogs and derivatives that may be used. The double displacement approach is highly applicable when dealing with the “epitope imprinting” method, when the binding agent:analyte conjugate is a synthetic peptide representing an epitope on the target analyte, against which a specific MIP was prepared, conjugated to the binding agent, such as biotin.

This double displacement approach has several advantages over the common single displacement method when the reporter is directly bound to the target molecule. As biotin is a relatively small molecule, adding it to the target molecule should not interfere much with its interaction with the analyte-specific MIP. The chemistry of biotin is well established and many commercial reagents are available for the modification of the target analytes. The binding of biotin to the various biotin-binding elements is very rapid and strong, contributing to the overall performance of the devices. Since the reporter is not bound directly to the analyte, there is great flexibility in the choice of the reporter and the ability to conjugate it to the biotin-derivative. The inability or difficulties in binding a reporter molecule to certain analytes may be a limiting factor for using the displacement approach in these cases. Additionally, it allows increasing the sensitivity of the devices, since various ways of signal amplification known to those skilled in the art may be employed due to the separation between the sensing elements and the results area.

Another advantage is that for devices for various analytes the invention employs the same signal formation method, most of the production is similar and only the specific MIP and the analyte conjugated to biotin would need to be developed for every individual product. This makes the time to market of new products shorter and reduces development and production costs.

If the whole protein is imprinted, the binding agent:analyte conjugate may optionally be composed of the target protein to which both binding agent and reporter molecules are conjugated. In this case, the conjugate may carry enough reporter molecules such that amplification might not be needed.

As used herein, the term ‘binding agent:analyte conjugate’ refers to the target analyte, conjugated to a binding agent (preferably biotin or a derivative of biotin) directly or via a spacer. Alternatively, the binding agent:analyte conjugate can optionally be a peptide corresponding to an epitope on the analyte conjugated to a binding agent. The binding agent has specific affinity to its corresponding binding partner. Where the binding agent is biotin and its derivatives, the corresponding binding partner or agent is Avidin, StrepAvidin, NeutrAvidin and similar biotin binding agents.

The first binding agent:analyte conjugate has high binding affinity to the analyte-specific MIP, but lower than the affinity of the unmodified analyte. The first binding agent:analyte conjugate molecules remain attached to the binding sites of the analyte-specific MIP even in a moist state unless they are displaced, in a dose-dependent manner, by the target analyte in the tested liquid sample. When displaced from the polymer, the first binding agent:analtye conjugate is freely mobile within the moist solid support.

The double displacement device further comprises a results zone, comprising a second binding agent:reporter conjugate binding element bound to the solid support on the flow path of the sample. The reporter-conjugate binding element has binding sites to which are attached a detectable, releasable, second binding agent:reporter conjugate.

As used herein, the term ‘binding element’ refers to a molecular structure able to specifically bind its respective binding partner with sufficient affinity. Neither the specific sequences nor the specific boundaries of such elements are critical, as long as binding activity is exhibited. Binding characteristics necessarily include a range of affinities, avidities and specificities, and combinations thereof, so long as binding activity is exhibited.

The binding agent:reporter conjugate comprises any reporter entity, as described above for the analyte analog:reporter conjugate, conjugated to a binding agent capable of specific binding to the same binding element as the binding agent:analyte conjugate.

The second binding agent possesses high affinity to this binding element, but lower than the affinity of the first binding agent:analyte conjugate that is employed in the device. Contact between the first binding agent:analyte conjugate and the binding element therefore causes displacement of the second binding agent:reporter conjugate. Displacement of the second binding agent:reporter conjugate is proportional to a concentration of the analyte in the liquid sample, such that the presence and/or intensity of the detectable signal is related to the amount of analyte in the sample.

When biotin, for example, is employed in the device as the binding agent in the binding agent:analyte conjugate, a derivative of biotin or other molecules (such as HABA and DTB) having lower affinity to biotin binding elements (such as Avidin, StrepAvidin NeutrAvidin and ExtrAvidin) than biotin are used in the construction of the binding agent:reporter conjugate.

As used herein, the term ‘binding partner’ or ‘binding agent’ refers to any molecule or composition capable of recognizing and binding to a specific structural aspect of another molecule or composition. Examples of such binding partners and corresponding molecule or composition include biotin/avidin, antigen/antibody, hapten/antibody, hormone/receptor, nucleic acid strand/complementary nucleic acid strand, substrate/enzyme, inhibitor/enzyme, protein A (or G)/immunoglobulins, carbohydrate/lectin, virus/cellular receptor and apoprotein/lipid.

The releasable first binding agent is selected from the group consisting of biotin, a biotin analog, a biotin derivative, an antigen, Protein A and Protein G, cellulose binding protein, hormones, toxins, lipids, fatty acids, complementary nucleic acid sequences, glycoconjugates, lectins, substrates and ligands.

The releasable second binding agent may be selected from the group consisting of a biotin analog, HABA, DTB, an antigen, Protein A and Protein G, cellulose binding protein, liposomes, hormones, toxins, lipids, fatty acids, complementary nucleic acids, glycoconjugates, lectins, substrates and ligands or their analogs, provided that said second binding agent has lower affinity to the reporter-conjugate binding element than said first binding agent. Biotin analogs are identified in Advances in Protein Chemistry, edited by Anfinsen, Edsall and Richards, Academic Press (1975), pages 104-111.

### Single Competition

The present invention further provide a diagnostic device wherein the sample application area comprises an analyte:analog conjugate, and the binding sites of the MIP are unoccupied. The conjugate may be provided, for example, in a pad, such that the conjugate become free-flowing once hydrated by the sample. In this case, the analyte analog:reporter conjugate competes with the analyte in the sample for the analyte-specific binding sites of the MIP. The unbound conjugate then migrates and is detected as described above for the displaced conjugate.

In yet a further aspect, the invention is directed to a method for detecting and determining the presence, absence or concentration of a target analyte present in a liquid sample by an assay comprising the steps of applying a sample suspected of containing the analyte to the sample application area of the competition type diagnostic device of the invention and allowing the sample to flow along or through the solid support and contact the MIP-conjugate zone so that, if analyte is present in the sample, analyte competes with the analyte analog:reporter conjugate for the binding sites of the MIP. the non-bound analyte analog:reporter conjugate flows to the results zone where it is captured by the analyte analog:reporter conjugate binding element, producing a detectable signal that indicates the presence or amount of the analyte in the sample.

### Double Competition

The present invention further provides a diagnostic device comprising a first binding agent:analyte conjugate, a second binding agent:reporter conjugate, and a second binding agent:receptor conjugate binding element. The first binding agent:analyte conjugate is optionally provided in the sample application area, and competes with the analyte for the analyte-specific binding sites of the MIP. Unbound first binding agent:analyte conjugate flows downstream, and competes with the second binding agent:reporter conjugate for binding to the second binding agent:receptor conjugate binding element.

In yet another aspect, the invention is directed to a method for detecting and determining the presence, absence or concentration of a target analyte present in a liquid sample by an assay, the method comprising the steps of:

(a) applying a sample suspected of containing the analyte to the sample application area of the double displacement diagnostic device of the invention; and

(b) allowing the sample to flow along or through the solid support and contact the MIP-conjugate zone so that, if analyte is present in the sample, analyte binds to the binding sites of the MIP displacing first binding agent:analyte conjugate which flows to the reporter-conjugate binding zone, the first binding area of reporter-conjugate binding element binding the displaced first binding agent:analyte conjugate and thereby displacing second binding agent:reporter conjugate which continues along the path of liquid flow to the results zone and is captured by the second binding area of reporter-conjugate binding element, producing a detectable signal that indicates the presence and/or amount of the analyte in the sample, wherein the amount of second binding agent:reporter conjugate captured is proportional to the concentration of said target analyte in the sample.

In another aspect, the invention is directed to a device comprising a solid support including a first component comprising a sample application pad impregnated with an analyte analog-conjugate comprising the analyte conjugated to biotin, optionally through a spacer (optionally, the analyte:analog conjugate may be impregnated in a dedicated reagent pad between the sample pad and the MIP); a second component comprising analyte-specific MIP, a third component containing a first biotin binding element saturated with reporter-conjugate comprising a biotin analog having lower affinity to the biotin binding element than biotin (such as desthiobiotin—DTB) conjugated to a reporter and a fourth component comprising a second biotin binding element

In yet another aspect, the invention is directed to a device comprising a solid support including a first component comprising an analyte-specific MIP saturated with an analyte analog-conjugate comprising the analyte conjugated to biotin, optionally through a spacer; a second component comprising a first biotin binding element saturated with reporter-conjugate comprising a biotin analog having lower affinity to the biotin binding element than biotin conjugated to a reporter, and a third component comprising a second biotin binding element.

### Competition Plus Displacement

The present invention may optionally further provide a diagnostic device and method comprising competition and displacement methods, in any order and combination.

For example, the method may comprise a first step wherein a first binding agent:analyte conjugate bound to the MIP is displaced by the analyte; and a second step wherein the displaced conjugate competes with a second binding agent:reporter conjugate for binding sites of a reporter conjugate binding element.

Alternatively, the first step may comprise competition between a first binding agent:analyte conjugate and the analyte for the binding sites of the MIP; and the second step may comprise displacement by the unbound analyte conjugate of a second binding agent:reporter conjugate from a reporter conjugate binding element.

In yet further aspects, the method includes the step of comparing the intensity of the signal in the results zone with the intensity of the signal in a reference zone to determine the concentration of the analyte in the sample.

In still further aspects, the results zone includes a scale parallel to the immobilized reporter-conjugate binding element calibrated to correspond to the analyte concentration in the liquid sample being analyzed and wherein the presence and concentration of analyte in the liquid is determined by the area covered by the reporter-conjugate along and through the reporter-conjugate binding element in the results zone.

In yet another aspect, the invention is directed to a method of detecting TSH present in a liquid sample, the method comprising the steps of:

a) contacting the sample with a competition, single or double displacement device according to the invention, and

b) detecting, in the results zone, the amount of reporter conjugate bound to the reporter-conjugate binding element, wherein the amount of the reporter-conjugate is indicative of the presence or amount of TSH in the sample;

c) diagnosing the activity of the thyroid.

The method of detecting TSH may be used for identification of an underactive thyroid gland (hypothyroidism) that can cause symptoms such as weight gain, tiredness, dry skin, constipation, a feeling of being too cold, or frequent menstrual periods; or an overactive thyroid (hyperthyroidism) that can cause symptoms such as weight loss, rapid heart rate, nervousness, diarrhea, a feeling of being too hot, or irregular menstrual periods.

In another aspect, the devices of the invention may be packaged together in a diagnostic kit with any or all of the diluents or reagents for extracting or processing the sample to elute the analyte and to detect a given analyte.

In yet other aspects, the diagnostic kit further includes packaging material and instructions for performance of the quantitative analysis on at least one type of liquid sample.

The sensitivity of particular assays is a function of the relative affinity and concentration of the various reagents, the times during which particular reagents and analyte are in contact with each other and the intensity of the signal produced by the reporting system. Those skilled in the art are familiar with methods for optimizing and characterizing the sensitivity and dose-response characteristics of conventional assays used in lateral flow and flow through devices.

Another important embodiment of the present invention is the method for visual monitoring and interpretation of the signal at the results window. In one variation of the monitoring system, termed the “vertical visualization method”, the reporter-conjugate binding element (any of a biotin-binding, or other reporter pair binding, element or analyte-reporter binding element) is placed vertical to the sample flow path to capture and concentrate as many as possible of the free-flowing reporter conjugate molecules, in a random order. The greater the amount of the reporter conjugate (which is a direct representation of the amount of the analyte in the tested sample) that binds the binding element, the stronger the visual signal obtained. Next to the results window is a reference window comprised of several indicator lines of binding element impregnated each with increasing precise amounts of the reporter conjugate. These lines will exhibit a range of color intensities, which are proportional to the known amount of the reporter conjugate bound to them. Comparison of the intensity of the signal in the results window with the intensity of the lines at the adjacent reference window enables good estimation of the amount of the analyte in the tested sample.

In another variation of the monitoring system, termed the “horizontal visualization method”, a stripe of well-defined reporter-conjugate binding element is accurately placed, horizontally, at the path of the sample flow. The device at this location should be manipulated in such a manner as to allow the flow of the liquid only through the area covered with the reporter-conjugate binding element and take measures to avoid sample flow around or below the binding element covered area. This can be achieved, for example, by physically cutting the porous material in such a way that only the binding element covered area remains in the flow path, or by using methods such as patterning of flow channels by photolithography [Martinez et al, 2007 Angwe. Chem. Int. 119, 1340-1342]. This ensures that all the conjugated reporter molecules displaced in response to the presence of the analyte in the sample arrive at the immobilized binding element. The displaced molecules of the reporter conjugate that reach the binding element migrate by capillary action and any reporter-conjugate molecules are captured by the binding element. However, as the absorption capacity of the binding element is limited, and as the binding sites of the binding element become filled with reporter-conjugate molecules, the binding element molecules at the front of the stripe becomes saturated and the reporter-conjugate molecules must migrate further down the stripe to find free binding sites. The higher the concentration of the analyte in the sample, the larger the area of the binding element that the reporter-conjugate will cover. Pre-calibration of the area covered by known amounts of the reporter-conjugate will enable accurate determination of the analyte concentration according to the distance covered by the reporter-conjugate along the stripe of the binding element by incorporating in the device a calibrated scale next to the immobilized binding element that allows the user to determine the amount of the analyte in the sample.

The devices may comprise more than one solid support, whereby more than one analyte can be detected in a sample.

The first binding area of reporter-conjugate binding element and the second binding area of reporter-conjugate binding element may be identical or different and each are selected from the group consisting of avidin, streptavidin, NeutrAvidin, ExtrAvidin compounds having high specific affinity to biotin, membranes, receptors, immunoglobulins, cellulose, enzymes, lectins, glycoconjugates, complementary nucleic acids and hydrophobic sites having high affinity to their respective binding partners.

### DETAILED DESCRIPTION OF THE FIGURES AND EXAMPLES

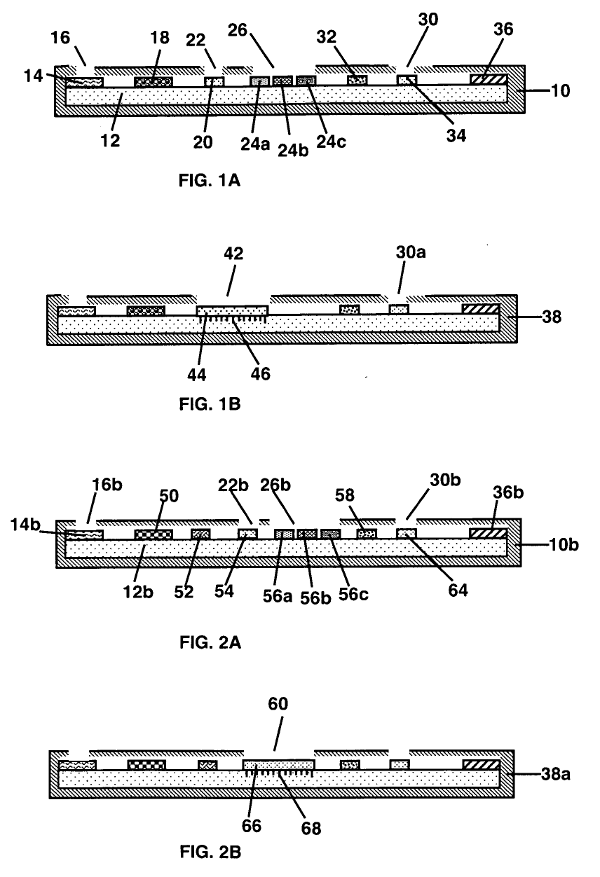

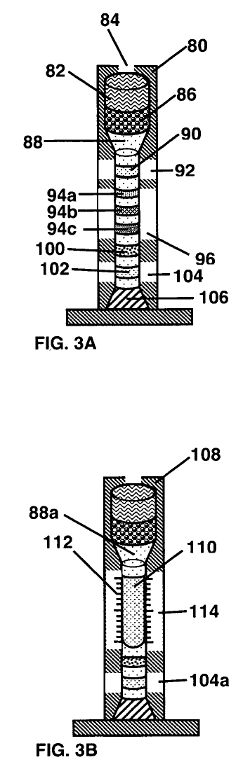

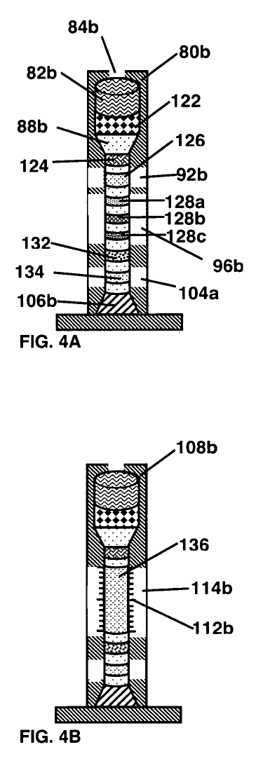

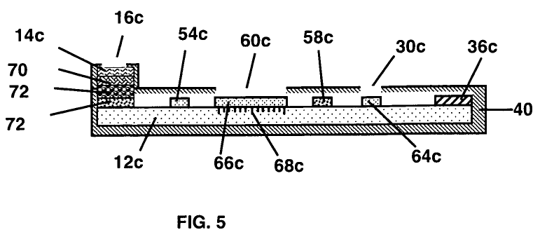

The following Examples are illustrative of the invention, and are not intended to be limiting in any way. One skilled in the art will recognize a variety of non-critical parameters that can be changed. The various components in the lateral flow devices are presented horizontally for clarity, but could be above each other or overlapping according to need. FIGS. 1-2 show four different embodiments of a lateral flow device of the present invention, FIGS. 3-4 show four different embodiments of a flow through device of the present invention and FIG. 5 show an embodiment of a device combining lateral flow and flow through elements, each of which will be described separately in Examples 1-9.

### Example I

### Embodiment of FIG. 1A

There is described below the structure and operation of a lateral flow device with competition or single displacement and vertical visualization.

FIG. 1A is a side view illustration of a lateral flow device for detecting and determining the presence, absence or concentration of a target analyte present in a liquid sample. The analytical test device comprises a hollow, solid casing 10 that contains a solid support 12 in the form of a porous test strip that serves as a carrier capable of conveying a liquid sample therethrough, the sample being movable along the solid support in the path of liquid flow by capillary action. The solid support 12 has defined zones, including a sample application area comprising a sample application pad 14 for applying the sample to the device and bringing it in contact with the solid support 12. The sample application pad 14 is located adjacent to an opening or window 16 in the casing 10 for applying the liquid sample. When competition type is employed the sample application pad 14 is impregnated with a releasable analyte analog:reporter conjugate in a dry state, alternatively a dedicated pad can be added.

The target analyte, if present in the liquid sample, is carried from the sample application area to a MIP-conjugate zone downstream of the sample application area comprising an analyte-specific MIP 18 fixed to the solid support 12 on the flow path of the sample. The MIP has analyte-specific binding sites saturated with a releasable analyte analog:reporter conjugate in a dry state, comprising the target analyte conjugated to a binding partner and to reporter molecules. The affinity of the analyte to the binding sites of the analyte-specific MIP is greater than the affinity of the analyte analog:reporter conjugate to the binding sites of the analyte-specific MIP. When the liquid sample containing the analyte contacts the analyte-specific MIP, the analyte in the sample binds to the analyte-specific cavities of the MIP and thereby displaces the analyte analog-reporter conjugate which occupy these cavities in an amount directly proportional to the concentration of the specific analyte, causing the displaced analyte analog:reporter conjugate to flow downstream in the path of liquid flow. In case of competition, the liquid sample dissolves the impregnated analyte analog:reporter conjugate and it is mixed with the analyte in the sample. When the liquid arrives to the analyte-specific MIP, the two molecules competes for the analyte-specific binding sites. An amount of analyte analog:reporter conjugate, proportional to the concentration of the specific analyte is left unbound and flow downstream in the path of liquid flow.

Further downstream on the solid support 12 is an analyte analog:reporter conjugate binding element that is the binding partner of the binding element bound to the analyte analog:reporter conjugate, 20 immobilized to the solid support 12 on the flow path of the sample downstream of the analyte-specific MIP 18. An opening in the casing 10 is located above the analyte analog:reporter conjugate binding partner 20, comprising the results window 22 of the device. The reporter conjugate binding partner 20 binds the unbound or displaced analyte analog:reporter conjugate displaced from the analyte-specific MIP 18 when a liquid sample containing the analyte flows in the flow path zone and provides a detectable signal in the results window 22 that indicates the presence or concentration of the analyte in the sample.

Further downstream on the solid support 12 are three discrete and non-overlapping indicator bands of analyte analog:reporter conjugate binding partner, each of which extends longitudinally on the strip, wherein 24a represents a low concentration reference band, 24b represents a medium concentration reference band and 24c represents a high concentration reference band. The three indicator bands comprise a reference zone for establishing a reference point in determining the presence or semi-quantification of an analyte in the tested sample. A corresponding reference window 26 appears in the casing 10 above the reference bands 24a, 24b and 24c. The reference zone may comprise at least one discrete band of binding element impregnated with a known quantity of the analyte analog:reporter conjugate in the case where the presence or absence of a target analyte in the sample is to be determined. The reference zone may comprise at least two spaced indicator bands of binding element impregnated each with increasing quantities of the analyte analog:reporter conjugate and exhibiting a range of color intensities proportional to the amount of the analyte analog:reporter conjugate bound to it, in the case where the amount of a target analyte in a sample is to be determined. The user interprets the results, and determines the presence, absence or semi-quantification of the concentration of the analyte in the sample, by visually comparing the intensity of the signal in the results window 22 with the intensity of one or more bands in the reference window 26.

Downstream of the reference bands 24a, 24b and 24c is a control pad 32 impregnated with analyte analog:reporter conjugate that is stationary in a dry state but becomes freely flowing when the solid support 12 is wetted with the liquid of the sample. Further downstream is another analyte analog:reporter conjugate binding partner 34 anchored to the solid support 12. There is a corresponding control window 30 in the casing above the analog:reporter conjugate binding partner 34 for viewing the results. The analyte analog:reporter conjugate that is released from the control pad 32 by the fluid from the sample reaches the binding partner 34 and become fixed to the solid support 12, producing a visual signal in the control window 30. The control pad and analyte analog:reporter conjugate binding element 34 together comprise a control zone for generating a positive control confirming the proper flow and binding of the analyte analog:reporter conjugate to the results zone to thereby determine that a test is working properly.

At the distal end of the device is an absorbent pad 36 of absorbent material in fluid communication with the solid support 12 when the pad 36 and solid support 12 are wet. The pad has sufficient porosity and capacity to absorb the surplus of the fluid and ensure continuous flow throughout the device.

### Example 2

### Embodiment of FIG. 1B

There is described below the structure and operation of a lateral flow device with competition or single displacement and horizontal visualization of the test results.

The lateral flow device is the same as that described above in Example 1, with similar numerals designating similar parts except that modifications are indicated with the reference numeral and the letter “a” affixed. The modifications are as follows: There is no reference zone in the device of FIG. 1B, thus, the casing 38 has fewer windows or openings, and has an enlarged results window 42. Results are read only from the results window 42 according to the distance that the analyte analog:reporter conjugate covered. The area covered by the immobilized analyte analog:reporter conjugate binding element 44 is enlarged with a scale 46 running parallel to it. The scale 46 and the area covered by the immobilized analyte analog:reporter conjugate binding element 44 are co-calibrated to correspond to the analyte concentration in the liquid sample being analyzed. The presence and concentration of analyte in the liquid is determined at the results window 42 by the area covered by the analyte analog:reporter-conjugate along and through the analyte:analog reporter-conjugate binding element 44. The results of the control signal are viewed in control window 30a.

### Example 3

### Embodiment of FIG. 2A

There is described below the structure and operation of a lateral flow device with competition/double displacement and vertical visualization of the test results.

FIG. 2A is a side view illustration of a lateral flow device for detecting and determining the presence, absence or concentration of a target analyte present in a liquid sample. The device comprises a hollow, solid casing 10b that contains a solid porous support 12b in the form of a test strip capable of conveying a liquid sample therethrough, the sample being movable along the solid support in the path of liquid flow by capillary action. The solid support 12b has defined zones, including a sample application area comprising a sample application pad 14b for applying the sample to the device and bringing it in contact with the solid support 12b. The sample application pad 14b is located adjacent to an opening or window 16b in the casing 10b for applying the liquid sample. When competition type is employed the sample application pad 14b is impregnated with a releasable analyte analog:reporter conjugate in a dry state, alternatively a dedicated pad can be added

The target analyte, if present in the liquid sample, is carried from the sample application pad 14b along and through a MIP-conjugate zone downstream of the sample application area 14b, the MIP-conjugate zone comprises an analyte-specific MIP 50 fixed to the solid support 12b on the flow path of the sample. The MIP 50 has analyte-specific binding sites saturated with a releasable first binding agent:analyte conjugate, which is the target, conjugated to a binding element, in a dry state. The affinity of the analyte to the binding sites of the analyte-specific MIP 50 is sufficiently greater than the affinity of the first binding agent:analyte conjugate to the binding sites of the analyte-specific MIP 50 to bring about the displacement of the first binding agent:analyte conjugate from the analyte specific binding sites of the MIP 50 in the presence of the analyte. When the liquid sample containing the analyte contacts the analyte-specific MIP 50, the analyte in the sample binds to the analyte-specific cavities of the MIP and thereby displaces the first binding agent:analyte conjugate which occupy these cavities in an amount directly proportional to the concentration of the specific analyte, causing the displaced first binding agent:analyte conjugate to flow downstream in the path of liquid flow. In case of competition, the liquid sample dissolves the impregnated analyte analog:reporter conjugate and it is mixed with the analyte in the sample. When the liquid arrives to the analyte-specific MIP, the two molecules compete for the analyte-specific binding sites. An amount of analyte analog:reporter conjugate, proportional to the concentration of the specific analyte is left unbound and flow downstream in the path of liquid flow

Further downstream on the solid support 12b is a reporter-conjugate binding zone that comprises a first binding area of reporter-conjugate binding element 52 immobilized to the solid support 12b on the flow path of the sample downstream of the analyte-specific MIP 50, comprising reporter-conjugate binding element 52 with its binding sites saturated with a detectable, releasable second binding agent:reporter conjugate in a dry state. The affinity of the first binding agent:analyte conjugate to the binding sites of the reporter-conjugate binding element 52 is greater than the affinity of the second binding agent:reporter conjugate to the binding sites of the reporter-conjugate binding element 52. The first binding area of reporter-conjugate binding element 52 binds the first binding agent:analtye conjugate and displaces the second binding agent:reporter conjugate in an amount directly proportional to the concentration of the specific analyte in the sample, causing the displaced second binding agent:reporter conjugate to continue to migrate downstream in the path of liquid flow. Alternatively, competition can be used and the second binding agent:reporter conjugate can be dried on a pad, rehydrated by the sample liquid and compete with the first binding agent:analyte conjugate for the binding sites at the first binding area. in this case, there is no need for greater affinity of the first binding agent:analyte to the binding sites of the reporter-conjugate binding element 52. The excess of unbound second binding agent:reporter conjugate will continue to migrate downstream in the path of liquid flow.

Further downstream on the solid support 12b is a second binding area of reporter-conjugate binding element 54 immobilized to the solid support 12b on the flow path of the sample downstream of the first binding area of reporter-conjugate binding element 52. An opening in the casing 10b is located above the second binding area of reporter-conjugate binding element 54, comprising the results window 22b of the device. The second binding area of reporter-conjugate-binding element 54 binds the second binding agent:reporter conjugate displaced from the first binding area of reporter-conjugate binding element 52 when a liquid sample containing the analyte flows in the flow path zone and provides a detectable signal that indicates the presence or concentration of the analyte in the sample.

As described with respect to the device in Example 1A, located further downstream on the solid support 12b are three discrete and non-overlapping indicator bands of the second binding agent:reporter conjugate binding element, wherein 56a represents a low concentration reference band, 56b represents a medium concentration reference band and 56c represents a high concentration reference band. The three indicator bands comprise a reference zone for establishing a reference point in determining the presence or semi-quantification of an analyte in the tested sample, above which is a corresponding reference window 26b in casing 10b. The user interprets the results, and determines the presence, absence or semi-quantification of the concentration of the analyte in the sample, by visually comparing the intensity of the signal in the results window 22b with the intensity of one or more bands in the reference window 26b.

As described above in connection with Examples 1A and 1B, downstream of the reference window 26b is a control pad 32b impregnated with second binding agent:reporter conjugate that is stationary in a dry state but becomes freely flowing when the solid support 12b is wetted with the liquid of the sample. Further downstream is another reporter-conjugate binding element 56 anchored to the solid support 12b. There is a corresponding control window 30a in the casing 10b above reporter-conjugate binding element 56 for viewing the results. The second binding agent:reporter conjugate that is released from the control pad 32b by the fluid from the sample reaches the binding element 56 and become fixed to the solid support 12b, producing a visual signal, viewed in the control window 30b. The control pad 32b and reporter-conjugate binding element 56 together comprise a control zone for generating a positive control.

At the distal end of the device is an absorbent pad 36b made of absorbent material in fluid communication with the solid support 12b when the pad 36b and solid support 12b are wet. The pad has sufficient porosity and capacity to absorb the surplus of the fluid and ensure continuous flow throughout the device.

### Example 4

### Embodiment of FIG. 2B

There is described below the structure and operation of a lateral flow device with competition/double displacement and horizontal visualization of the test results. The lateral flow device is the same as that described above in Example 3, with similar numerals designating similar parts except that modifications are indicated with the reference numeral and the letter “a” affixed. The modifications are as follows: There is no reference zone in the device of FIG. 2B. Results are read only from the results window 60 according to the distance that the second binding agent:reporter conjugate covered. The second binding area of reporter-conjugate binding element 66 under the results window 60 in casing 38a is elongated and includes a scale 68 parallel to the immobilized second binding area of reporter-conjugate binding element 66 calibrated to correspond to the analyte concentration in the liquid sample being analyzed. The presence and concentration of analyte in the liquid is determined by the area covered by the second binding agent:reporter-conjugate along the second binding area of reporter-conjugate binding element 66 as viewed in the results window 60.

### Example 5

### Embodiment of FIG. 3A

There is described below the structure and operation of a flow through device with competition or single displacement and vertical visualization.

FIG. 3A is a side view illustration of a flow through device for detecting and determining the presence, absence or concentration of a target analyte present in a liquid sample. The device comprises a hollow, solid casing 80 containing defined layers or zones made of reagent-containing porous materials, arranged so that fluid that is applied to the top of the device flows vertically through the various layers of the device, from one layer to another, until the fluid contacts an absorbent material at the bottom of the device.

A sample application area comprising a sample application pad 82 for applying the sample to the device is located adjacent to an opening or window 84 in the casing 80 at the top of the device and leads to a MIP-conjugate zone comprising an analyte-specific MIP 86 having analyte-specific binding sites saturated with a releasable analyte analog:reporter conjugate in a diy state. The analyte analog:reporter conjugate may comprise also polypeptide or protein corresponding to the target analyte, conjugated to both binding partner and reporter molecules. The affinity of the analyte to the binding sites of the analyte-specific MIP 86 is greater than the affinity of the analyte analog:reporter conjugate to the binding sites of the analyte-specific MIP 86. In case of a competition type device, the application pad, or a dedicated reagent pad bellow it is impregnated with the analyte analog:reporter conjugate, while the MIP 86 have its binding sites free. in this case, difference in affinity to the MIP is not mandatory. The target analyte, if present in the liquid sample, migrates from the sample application pad 82 through the analyte-specific MIP 86. When the liquid sample containing the analyte contacts the analyte-specific MIP 86, the analyte in the sample binds to the analyte-specific cavities of the MIP and thereby displaces the analyte analog-reporter conjugate which occupy these cavities in an amount directly proportional to the concentration of the specific analyte in the sample, causing the displaced analyte analog:reporter conjugate to migrate further down through the device. For a competition type device, the liquid of the sample dissolves the analyte analog:reporter conjugate from the sample pad or the dedicated reagent pad and the analyte analog:reporter conjugate is mixed together with the analyte in the sample and flow together until the MIP zone 86 where they compete for the analyte-specific binding sites. An amount of analyte analog:reporter conjugate, directly proportional to the concentration of the specific analyte in the sample is left unbound and migrate further down stream the device.

Next is an analyte analog:reporter conjugate binding element 90, which specifically binds its binding partner conjugated to the analyte analog:reporter conjugate, fixated to the porous carrier or support 88 on the flow path of the sample downstream of the analyte-specific MIP 86. A clear window in the solid casing 80 is located in front of the analyte analog:reporter conjugate binding element 90, comprising the results window 92 of the device. The unbound analyte analog:reporter conjugate or the analyte analog:reporter conjugate displaced from the analyte-specific MIP 86 by the analyte migrate to the analyte analog:reporter conjugate binding element 90 where they are captured and provide a detectable signal, indicatin the presence or concentration of the analyte in the sample in the results window 92.

The next zone is a reference zone comprising three discrete and non-overlapping indicator bands of analyte analog:reporter conjugate binding element, separated by porous carrier zones 88, wherein 94a represents a low concentration reference band, 94b represents a medium concentration reference band and 94c represents a high concentration reference band. A clear reference window 96 is located in front of the three indicator bands 94a, 94b and 94c. The user interprets the results by visually comparing the intensity of the signal in the results window 92 to the intensity of the bands in the reference window 96.

Underneath the reference zone is a porous carrier zone 88 followed by a control area 100 comprising a porous carrier impregnated with unbound analyte analog:reporter conjugate. This conjugate 100 is stationary in the dry state but becomes freely flowing when the porous carrier is wetted with the liquid of the sample. Further down, separated by another porous carrier zone 88, is another analyte analog:reporter conjugate binding element 102. A control window 104 is located in front of the reporter conjugate binding element 102. The analyte analog:reporter conjugate which is released from the control area 100 by the fluid from the sample reaches the analyte analog:reporter conjugate binding element 102 and is bound to the carrier, producing a visual signal visible at the control window 104. At the distal end of the device is an absorbent material 106 that absorbs the surplus of the fluid and ensures continuous flow throughout the device.

### Example 6

### Embodiment of FIG. 3B

There is described below the structure and operation of a flow through device with competition or single displacement and horizontal visualization of the test results.

The flow through device is the same as that described above in Example 5, with similar numerals designating similar parts, with minor modifications as follows: There is no reference zone in the casing 108 of the device of FIG. 3B. Results are read only from the results window 114 according to the distance that the analyte analog:reporter conjugate covered. The analyte analog:reporter-conjugate binding element 110 as well as the results window 114 are elongated. A scale 112 runs parallel to the immobilized analyte analog:reporter-conjugate binding element 110, both co-calibrated to correspond to the analyte concentration in the liquid sample being analyzed. The presence and concentration of analyte in the liquid is determined by the area covered by the analyte analog:reporter-conjugate through the analyte:analog reporter-conjugate binding element that is seen in the results window 114. The control results are viewed in the control window 104a.

### Example 7

### Embodiment of FIG. 4A

There is described below the structure and operation of a flow through device with competition/double displacement and vertical visualization of the test results.

FIG. 4A is a sectional side view illustration of a flow through device for detecting and determining the presence, absence or concentration of a target analyte present in a liquid sample using competition/double displacement. The device comprises a hollow, solid casing 80b containing defined layers or zones made of reagent-containing porous materials, arranged so that fluid that is applied to the top of the device flows vertically through the various layers of the device, from one layer to another, until the fluid contacts an absorbent material at the bottom of the device.

A sample application pad 82b for applying the sample to the device is located adjacent to an opening or window 84b in the casing 80b at the top of the device and leads to a MIP-conjugate zone comprising an analyte-specific MIP 122 having analyte-specific binding sites saturated with a releasable first binding agent:analyte conjugate in a dry state. The first binding agent:analyte conjugate may comprises a synthetic peptide corresponding to the epitope of the target that was imprinted, conjugated to a binding partner. The affinity of the analyte to the binding sites of the analyte-specific MIP 122 is greater than the affinity of the first binding agent:analyte conjugate to the binding sites of the analyte-specific MIP 122. The target analyte found in the liquid sample migrates from the sample application pad 82 through the MIP-conjugate zone where it binds to the analyte-specific cavities of the MIP 122 and thereby displaces the first binding agent:analyte conjugate which occupy these cavities in an amount directly proportional to the concentration of the specific analyte, causing the displaced first binding agent:analyte conjugate to migrate further down through the device. Alternatively, if competition type device is used, the application pad 82b, or a dedicated reagent pad is impregnated with the releasable first binding agent:analyte conjugate. For a competition type device, the liquid of the sample dissolves the analyte analog:reporter conjugate from the sample pad or the dedicated reagent pad and the analyte analog:reporter conjugate is mixed together with the analyte in the sample and flow together until the MIP zone 122 where they compete for the analyte-specific binding sites. an amount of analyte analog:reporter conjugate, directly proportional to the concentration of the specific analyte in the sample is left unbound and migrate further down stream the device.

The next layer is a porous carrier or support 88b, followed by a layer of a reporter-conjugate binding zone comprising a first binding area of reporter-conjugate binding element 124 having binding sites saturated with a detectable, releasable second binding agent:reporter conjugate in a dry state. This binding element has specific affinity to its binding partners conjugated both to the first binding agent:analyte as well as to the second binding agent:reporter conjugate. The affinity of the first binding agent:analyte conjugate to the binding sites of the reporter-conjugate binding element 124 is greater than the affinity of the to the binding sites of the reporter-conjugate binding element 124. The first binding area of reporter-conjugate binding element 124 in the reporter conjugate binding zone binds the first binding agent:analtye conjugate and displaces the second binding agent:reporter conjugate in an amount directly proportional to the concentration of the specific analyte in the sample, causing the displaced (displacement) or unbound (competition) second binding agent:reporter conjugate to continue to migrate further down the device. Alternatively, competition can be used and the second binding agent:reporter conjugate can be dried prior to the binding area of reporter-conjugate binding element 124, rehydrated by the sample liquid and compete with the first binding agent:analyte conjugate for the binding sites at the first binding area. In this case, there is no need for greater affinity of the first binding agent:analyte to the binding sites of the reporter-conjugate binding element 124. The excess of unbound second binding agent:reporter conjugate will continue to migrate downstream in the path of liquid flow.

Next is a second binding area of reporter-conjugate binding element 126 fixated to a porous carrier. An opening in the casing 80b is located in front of reporter-conjugate binding element 126, comprising the results window 92b of the device. The second binding area of reporter-conjugate-binding element 126 binds the unbound or displaced second binding agent:reporter conjugate coming from the analyte-specific MIP 122 when a liquid sample containing the analyte migrates to reporter-conjugate binding element 126, and provides a detectable signal in the results window 92b that indicates the presence or concentration of the analyte in the sample.

The next zone is the reference zone comprising three discrete and non-overlapping indicator bands of second binding agent:reporter conjugate binding element, wherein 128a represents a low concentration reference band, 128b represents a medium concentration reference band and 128c represents a high concentration reference band. A clear reference window 96b is located in front of the reference bands. The user interprets the results by visually comparing the intensity of the signal in the results window 92b to the intensity of the bands in the reference window 96b.

Underneath the reference zone is a control zone comprising a porous carrier 132 impregnated with unbound second binding agent:reporter conjugate. This conjugate is stationary in the dry state but becomes freely flowing when the porous carrier is wetted with the liquid of the sample. Further down is another reporter-conjugate binding element 134, visible at the control window 104b. The second binding agent:reporter conjugate which is released from the control pad 132 by the fluid from the sample reaches the reporter conjugate binding element 134 and is bound to the carrier, producing a visual signal at the control window 104b. At the distal end of the device is an absorbent material 106b that absorbs the surplus of the fluid and ensures continuous flow throughout the device.

### Example 8

### Embodiment of FIG. 4B

There is described below the structure and operation of a flow through device with competition/double displacement and horizontal visualization of the test results. The flow through device is the same as that described above in Example 7, with similar numerals designating similar parts, except that modifications are indicated with the reference numeral and the letter “b” affixed. The modifications are as follows: There is no reference zone in the casing 108b of the device of FIG. 4B. Results are read only from the results window according to the distance that the second binding agent:reporter conjugate covered. The immobilized second binding agent:reporter conjugate binding element 136 as well as the results window 114b are elongated. A scale 112b runs parallel to the immobilized second binding agent:reporter conjugate binding element 136 co-calibrated to correspond to the analyte concentration in the liquid sample being analyzed. The presence and concentration of analyte in the liquid is determined by the area covered by the second binding agent:reporter conjugate through the second binding agent:reporter conjugate binding element 136 that is viewed in the results window 114.

### Example 9

### Embodiment of FIG. 5

There is described below the structure and operation of a combined lateral flow/flow through device with competition and vertical visualization of the test results.

FIG. 5 is a side view illustration of a lateral flow device for detecting and determining the presence, absence or concentration of a target analyte present in a liquid sample. The device comprises a hollow, solid casing 40 that contains designated porous pads; sample pad 14c, conjugate pad 70 impregnated with releasable first binding agent:analyte conjugate, MIP pad 72 comprises analyte-specific MIP and reagent reporter pad 74 impregnated with second binding agent:reporter conjugate, all stacked in close contact one on top of the other, pad 74 being in close contact with a solid porous support 12c. The stacked pads and following test strip capable of conveying a liquid sample therethrough, the sample being movable through the pads and along the solid support in the path of liquid flow by gravity and capillary action. The sample application pad 14c is located adjacent to an opening or window 16c in the casing 40 for applying the liquid sample. The target analyte, if present in the liquid sample, is carried from the sample application pad 14c through the conjugate pad 70 where the liquid sample dissolves the impregnated releasable first binding agent:analyte conjugate and it is mixed with the analyte in the sample. When the liquid flow downwards to the analyte-specific MIP located at MIP zone 70, the two molecules compete for the analyte-specific binding sites. An amount of first binding agent:analyte conjugate, proportional to the concentration of the specific analyte in the sample is left unbound and flow downstream in the path of liquid flow. The unbound first binding agent:analyte conjugate continues flowing with the sample liquid to the reporter reagent pad 74 where the liquid dissolves the second binding agent:reporter conjugate. The liquid containing the unbound first binding agent:analyte conjugate and the second binding agent:reporter conjugate flows through pad 74 and come into contact the solid porous support 12c, which convey the flow of the liquid further downstream to the first reporter-conjugate binding zone 54c comprising reporter-conjugate binding element immobilized to the solid support 12c. The first binding agent:analyte conjugate and the second binding agent:reporter conjugate competes for the binding sites of the first reporter-conjugate binding zone, living unbound second binding agent:reporter conjugate. The unbound second binding agent:reporter conjugate to continue to migrate downstream in the path of liquid flow

Further downstream on the solid support 12c is a second binding area of reporter-conjugate binding element 60c immobilized to the solid support 12c on the flow path of the sample The second binding area of reporter-conjugate binding element 66c under the results window 60c in casing 40 is elongated and includes a scale 68c parallel to the immobilized second binding area of reporter-conjugate binding element 66c calibrated to correspond to the analyte concentration in the liquid sample being analyzed. The presence and concentration of analyte in the liquid is determined by the length of the area covered by the second binding agent:reporter-conjugate along the second binding area of reporter-conjugate binding element 66 as viewed in the results window 60.

There is a corresponding control window 30c in the casing 40 above reporter-conjugate binding element 64c for viewing the results. The second binding agent:reporter conjugate that is released by the sample liquid from control pad 58c reaches the binding element 64c and become fixed to the solid support 12c, producing a visual signal, viewed in the control window 30c. The control pad 58c and reporter-conjugate binding element 64c together comprise a control zone for generating a positive control.

At the distal end of the device is an absorbent pad 36c made of absorbent material in fluid communication with the solid support 12c. The pad has sufficient porosity and capacity to absorb the surplus of the fluid and ensure continuous flow throughout the device.

### Example 10

### Materials and Methods

The following materials and methods were used in the examples described below. A. Porous, solid support: The porous, solid support used in the rapid diagnostic device of the invention is a membrane filter comprising a strongly adsorptive substance having a large surface area, such as PuraBind™ (Whatman, USA) which is 100% nitrocellulose with no post-manufacture treatments.

B. Preparation of synthetic peptide representing epitope on the beta subunit of TSH: The amino acid sequence of the target epitope (LSCKCGKCNTDY) was obtained from a paper that described binding sites of antibodies on the beta subunit of TSH (Fairlie et al, 1995, Biochem. J. 308, 203-210). The peptide was prepared using an automated 433A peptide synthesizer (Applied Biosystems, USA).

C. Preparation of TSH-specific MIP: Preparation was in accordance with the methods set forth in the review by Yan and Row (Int. J. Mol. Sci. 2006, 7, 155-178) as follows: The functional monomer, Methacrylic acid (MAA) (Cat. No. 155721, Aldrich) is mixed with the target print molecule, in this case the synthetic peptide described above, together with the cross-linking monomer ethylene glycol dimethacrylate (EGDMA), (Cat. No. 33568-1, Aldrich) in 3% water in acetonitrile (Cat. No. 360457, Sigma-Aldrich) together with the initiator 2,2′-azobis(2,4-dimethylvaleronitrile) (Cat. No. 002094, Chemos GmBH, Germany). The mixture is degassed and purged with nitrogen for 5 min and the polymerization takes place following for 16 hours at 40° C., resulting with a rigid insoluble polymer with TSH-specific binding cavities present within the polymeric network. The bulk polymer is ground in a mechanical mortar and wet sieved in water through a 25 μm sieve. The print molecule is extracted by extensive washing of the particles with methanol-acetic acid (9/1, v/v), the polymer particles dried under vacuum and stored desiccated.

D. Preparation of TSH peptide-biotin conjugate: The synthetic peptide was conjugated to biotin using the PeptiTag—Biotin kit (BioSight, Israel).

E. Preparation of the MIP-Conjugate Zone: The binding cavities of the TSH-specific MIP are first saturated with the TSH peptide-Conjugate (i.e., peptide-biotin conjugate) by incubation of the sieved TSH-specific MIP particles with a solution of the TSH peptide-biotin conjugate for 24 hours at 37° C. followed by washing to remove the excess conjugate. The washed MIP particles are then dried at 60° C. in an oven and packaged inside filter paper bags, manufactured by Filtech Fabrics Ltd, India, similar to those used as tea bags. The bag containing the loaded MIP particles is attached to the nitrocellulose membrane at the MIP-conjugate zone by pressure-sensitive adhesive-coated films (Cat. No. ARcare® 8570, Adhesives Research, U.S.).

F. Preparation of the Reporter-Conjugate: As the reporter-conjugate molecule, 4-hydroxyazobenzene-2-carboxylic acid (HABA) (Cat. No. 54791, Fluka), conjugated to BSA (Cat. No. A3902, Sigma) coated with colloidal gold particles, is used. The HABA is attached to BSA in accordance with the method described by Hofstetter et al., (Analytical Biochemistry (2000) 284, 354-366). HABA azo-dye binds to the biotin-binding site of avidin with an affinity constant of Kd=10⁻⁶ M. HABA is displaced from this binding site by biotin which has an affinity constant of Kd=10⁻¹⁵. Gold sol is available from BioAssay Works MD, USA. Loading of HABA-BSA conjugate with gold sol is performed using the coupling method described by Horrisberger and Clerc (Histochemistry, 1985, 82, 219-223A).

G. For both biotin-binding element containing zones, NeutrAvidin™ Biotin-Binding Protein (Pierce, USA) is preferably employed. This protein is an excellent alternative to other biotin-binding proteins, such as avidin or streptavidin, when nonspecific binding must be minimized. Its immobilization was performed by direct blotting of the NeutrAvidin to the nitrocellulose membrane. 20 μg/ml NeutrAvidin in 0.5M phosphate buffer, pH 7.2 with 0.5M NaCl, was incubated O.N. at room temperature and washed once with PBS and air dried. The prepared membrane was stored desiccated at room temperature until the final construction of the device.

H. Preparation of TSH-reporter:binding partner conjugate (Biotin-TSH-Gold) is accomplished by attaching biotin to the TSH beta subunit using EZ-Link Sulfo-NHS-Biotin labeling kit. (Pierce, USA) The TSH-Biotin conjugate is then coated with gold sol. The coating is done as described earlier for the coating of the BSA with the gold sol. When in free mobile form, this conjugate is captured by the immobilized NeutrAvidin, and a visual signal is obtained due to the gold particles covering the molecule.

I. Preparation of TSH-reporter conjugate binding zone: The TSH-reporter conjugate binding zone is comprised of a zone on the nitrocellulose solid support having NeutrAvidin immobilized to it (as described above for the biotin-binding element zones).

J. Preparation of reference zone: The reference zone is preferably comprised of five identical parallel bands of TSH-reporter conjugate binding zones. Each band is applied accurately with a solution of the biotin-TSH-Gold conjugate. Eventually, the five bands have 0.2, 1.0, 3.0, 5.0 and 7.5 mIU/L of the conjugate bound to them, respectively. By comparing the color intensity of band in the results window with the reference bands, the user may determine the range of the amount of TSH in the tested sample.

### Example 11

### Assay for TSH Using a Lateral Flow Device (Double Displacement)

There is described below the structure and operation of a particular displacement assay of the invention.

### Structure:

A device as shown in FIG. 2A is used to quantify TSH in liquid samples obtained from humans. The device comprises a housing containing windows that exposes areas of the solid support for viewing. The device includes a sample application area comprising a pad (LF1, Whatman, USA) to which a liquid sample of whole blood is introduced, bringing the sample fluid in contact with a solid support (test strip) of porous membrane (PuraBind™, Whatman, USA). The pad is designed to separate the plasma from the blood cells. The solid support comprises a defined MIP-conjugate zone comprising TSH-specific MIP saturated with TSH-peptide-biotin conjugate. Downstream of the MIP-conjugate zone is a reporter-conjugate binding zone comprising NeutrAvidin impregnated with HABA conjugated to BSA coated with gold sol. Further downstream is a results zone comprising immobilized NeutrAvidin with a scale to be used as an aid in quantifying the concentration of MMA in the sample, followed by a control pad comprising dried HABA-BSA-GOLD (reporter-conjugate) and a control zone comprising NeutrAvidin immobilized to the solid support.

### Operation

To initiate the assay, a drop of whole blood is applied to the sample application area. The plasma leaving the sample application area contacts the nitrocellulose membrane and flows along the device by capillary action to contact the TSH-specific MIP zone, which is impregnated with TSH-peptide-biotin conjugate. As the liquid front moves through the MIP-conjugate zone, the TSH present in the sample displaces molecules of TSH-peptide-biotin conjugate from the MIP, in an amount proportional to its concentration.

The displaced TSH-peptide-biotin conjugate molecules flow downstream in the fluid, reaching the reporter-conjugate binding zone that comprises HABA-BSA-Gold reporter-conjugate, immobilized to NeutrAvidin. The biotin of the TSH-peptide-biotin conjugate displaces the reporter-conjugate from the NeutrAvidin, in an amount proportional to the amount of the displaced TSH-peptide-biotin conjugate, due to the higher affinity of biotin.

The displaced reporter-conjugate migrates further downstream until it comes in contact with the NeutrAvidin binding element at the results zone. Parallel to the NeutrAvidin binding element is a reference scale titrated to allow interpretation of the amount of TSH in the sample by determining the distance traveled by the reporter-conjugate along the binding element at the results window. A visual signal is evident in the results zone allowing the user to determine the amount of TSH in the blood sample. The results are determined about 15 minutes after application of the sample, which is the time required for ensuring the proper functioning of all components of the device.

After passing the results zone, the fluid sample continues to move laterally across the control pad, bringing the reporter-conjugate namely HABA-BSA-Gold impregnated in the control pad into free flowing condition along the device until the NeutrAvidin band located in the control zone, where it is captured. A visual line forms across the entire control zone indicating that the assay has functioned properly. The excess liquid and reagents will continue to move laterally across the device and collect in the absorbent pad.

### Example 12

### Assay for TSH Using a Diagnostic Flow Through Device (Double Displacement)

There is described below the structure and operation of a particular displacement assay of the invention (column-type assembly).

### Structure

A device as shown in FIG. 4B is used to quantify TSH in liquid samples obtained from humans. The device comprises a housing containing windows that exposes areas of the solid support for viewing. The device includes a sample application area comprising a pad designed to separate the plasma from the blood cells (LF1, Whatman, USA) to which a liquid sample of whole blood is introduced, bringing the fluid sample in contact with a zone of porous solid support comprising unmodified beads (Sigmacell® Cellulose, Type 50, Cat. No S5504 Sigma).

Further downstream is a MIP-conjugate zone comprising packed TSH-specific MIP particles saturated with TSH-peptide-biotin conjugate located in physical contact underneath the solid support zone. Further downstream, in physical contact with the MIP-conjugate zone is a reporter-conjugate binding zone comprising packed cellulose biotin-immobilized sepharose beads (Cat. No VIT-H-4S, Affiland S.A. Belgium) coated with NeutrAvidin saturated with HABA-BSA-GOLD. Further downstream, in physical contact with the cellulose beads, is a results zone comprising packed biotin-immobilized sepharose beads coated with NeutrAvidin, separated by a zone of unmodified beads. The chromatographic flow of the molecules through the beads helps to form a distinct and concentrated zone of the analyte in the fluid. Parallel to the results zone is a scale, visible in a viewing window, to be used as an aid in quantifying the concentration of the TSH in the sample, followed by a control pad comprising dried HABA-BSA-Gold (reporter-conjugate) and a control zone comprising biotin-immobilized sepharose beads coated with NeutrAvidin. Sample is introduced at the sample application area and following analysis of a sample in the device, a visual signal is evident in the results zone and in the control zones, allowing the user to determine the amount of TSH in the blood sample, as well as validating the proper performance of the device.

### Operation:

To initiate the assay, a drop of whole blood is applied to the sample application area at the top of the device. The plasma leaving the sample application area flows along the device by capillary action to contact the TSH-specific MIP zone, which is impregnated with TSH-peptide-biotin conjugate. As the liquid front moves through the MIP-conjugate zone, the TSH present in the sample displaces molecules of TSH-peptide-biotin conjugate from the MIP, in an amount proportional to its concentration.

The displaced TSH-peptide-biotin conjugate molecules flow down the device in the fluid, reaching the reporter-conjugate binding zone. The biotin of the TSH-peptide-biotin conjugate displaces the reporter conjugate from the NeutrAvidin, in an amount proportional to the amount of the displaced TSH-peptide-biotin conjugate due to the higher affinity of biotin.

The displaced reporter conjugate migrates down the device until it comes in contact with the NeutrAvidin coated cellulose beads at the results zone. Parallel to the NeutrAvidin containing binding element is a reference scale, titrated to allow interpretation of the amount of the TSH in the sample by determining the distance traveled by the reporter-conjugate along the binding element at the results window. The results are determined about 15 minutes after the sample application, which is the time required to ensure the proper functioning of all components of the device.

After passing the results zone, the fluid sample continues to move down to the control pad bringing the un-bound reporter-conjugate, namely, HABA-BSA-GOLD impregnated in the control pad into free flowing condition down the device until the NeutrAvidin coated beads located at the control zone. A visual line forms across the entire control zone indicating that the assay has functioned properly. The excess liquid and reagents will continue to move down the device and collect in the absorbent pad.

### Example 13

### Fluorescent Displacement Assay Kit for the Determination of B-Type Naturietic Peptide (BNP)

### Structure:

This device comprises a portable detection unit. For the sake of convenience, this description will refer to the general structure of lateral flow device described in Examples 1-4, it being understood that the device may take the form of a flow-through device as described in Example 5-8, with minor modifications.

The device includes a sample application area comprising a pad designed to separate the plasma from the blood cells (LF1, Whatman, USA) to which a liquid sample of whole blood is introduced, bringing the fluid sample in contact with the nitrocellulose membrane. The MIP-conjugate zone (detection zone) comprises a BNP-peptide-specific MIP, saturated with BNP-peptide-biotin conjugate. The reporter-conjugate is HABA-BSA-coated with fluorescent dye molecules (HABA-BSA-FLUROFOR). The reporter molecule of the reporter-conjugate is the fluorescent dye Alexa Fluor 488 (Invitrogen, USA) and determination of the results is done by means of a handheld portable fluorescent assay reader (ESE; Stockach, Germany). The device is built with a casing designed to fit into the fluorescent reader, in such a way that the area corresponding to the results zone in the visual devices is aligned with the fluorescent detection unit of the reader.

### Operation:

The device for detecting BNP is used to monitor the presence and concentration of the BNP in serum. Prior to application of the serum sample, the device is fitted into the reader which activates the instrument and brings it to a stand-by position (only proper fitting activates the instrument or else an error message appears). The sample application triggers the instrument to perform a count-down that results in the measurement of the fluorescence intensity about 15 minutes after the sample application, which is the time required to ensure the proper function of all the components of the assay.

The BNP in the sample displaces the BNP-peptide-biotin from the BNP-peptide-specific MIP and the displaced atrazine-biotin in turn displaces the HABA-BSA-fluor dye from the NeutrAvidin in the reporter-conjugate binding zone. The reporter-conjugate migrates downstream until the result zone, where it is captured by the NeutrAvidin. The amount of BNP in the sample is determined by the fluorescent reader by comparing the signal obtained from the sample to that of an internal calibration curve. The results are displayed on the LCD of the instrument in pg/ml (with a limit of detection of 10 pg/ml).

### Example 14

### Assay for MMA Using a Lateral Flow Device (Competition/Displacement)

There is described below the structure and operation of a particular displacement assay of the invention.

### Structure:

A device as shown in FIG. 2B is used to quantify MMA in liquid samples obtained from humans. The device comprises a housing containing windows that exposes areas of the solid support for viewing. The device includes a sample application area comprising a pad (LF1, Whatman, USA) impregnated with to which MMA-biotin conjugate, to which a liquid sample of whole blood is introduced, bringing the sample fluid in contact with a solid support (test strip) of porous membrane (PuraBind™, Whatman, USA). The pad is designed to separate the plasma from the blood cells. The solid support comprises a defined MIP zone comprising MMA-specific MIP. Downstream of the MIP zone is a reporter-conjugate binding zone comprising NeutrAvidin impregnated with desthiobiotin (DTB) attached to colored polystyrene beads (K1-030 bleu(39457), Merck—Estapor, France). Further downstream is a results zone comprising immobilized NeutrAvidin with a scale to be used as an aid in quantifying the concentration of MMA in the sample, followed by a control pad comprising dried DTB-beads (reporter-conjugate) and a control zone comprising NeutrAvidin immobilized to the solid support.

### Operation

To initiate the assay, a drop of whole blood is applied to the sample application area and dissolves the impregnated MMA-biotin conjugate. The plasma containing the conjugate leaves the sample application area contacts the nitrocellulose membrane and flows along the device by capillary action to contact the MMA-specific MIP zone. As the liquid front moves through the MIP-conjugate zone, the MMA present in the sample competes with the molecules of MMA-biotin conjugate for the MMA-specific binding sites of the MIP, and due to its superior affinity to these sites an amount of conjugate, proportional to its concentration in the sample is left unbound.

The unbound MMA-biotin conjugate molecules flow downstream in the fluid, reaching the reporter-conjugate binding zone that comprises DTB-beads reporter-conjugate, immobilized to NeutrAvidin. The biotin of the MMA-biotin conjugate displaces the reporter-conjugate from the NeutrAvidin, in an amount proportional to the amount of the displaced MMA-biotin conjugate, due to the higher affinity of biotin. The displaced reporter-conjugate migrates further downstream until it comes in contact with the NeutrAvidin binding element at the results zone. Parallel to the NeutrAvidin binding element is a reference scale, titrated to allow interpretation of the amount of MMA in the sample by determining the distance traveled by the reporter-conjugate along the binding element at the results window. A visual signal is evident in the results zone allowing the user to determine the amount of MMA in the blood sample. The results are determined about 15 minutes after application of the sample, which is the time required to ensure the proper functioning of all components of the device.

After passing the results zone, the fluid sample continues to move laterally across the control pad, bringing the reporter-conjugate namely DTB-beads impregnated in the control pad into free flowing condition along the device until the NeutrAvidin band located in the control zone, where it is captured. A visual line forms across the entire control zone indicating that the assay has functioned properly. The excess liquid and reagents will continue to move laterally across the device and collect in the absorbent pad.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples, which are intended to illustrate but not to limit the invention.

Although the invention has been described in conjunction with specific embodiments thereof, it is evident that many alternatives, modifications and variations will be apparent to those skilled in the art, such as addition of reagents to control the conditions of the assay, the pH for example. Accordingly, it is intended to embrace all such alternatives, modifications and variations that fall within the spirit and broad scope of the appended claims.

What is claimed is:

1. A diagnostic device for detecting at least one analyte in a liquid sample, the device comprising:
a) a molecular imprinted polymer having analyte-specific binding sites fixed to a solid support, wherein the liquid sample is movable along a flow path along said solid support;
b) a sample application area for applying the liquid sample to the device and bringing it in contact with said molecular imprinted polymer; and
c) a detection zone for detecting binding of said analyte to said molecular imprinted polymer.

2. The device of claim 1, wherein the device comprises a lateral flow device, a flow through device, or a device combining lateral flow and flow through elements, wherein said liquid sample flows from said sample application area along or through said solid support components of the device along a flow path of the device.

3. The device of claim 1, wherein the analyte displaces an analyte analog:reporter conjugate releasably bound to said molecular imprinted polymer, wherein displacement of said analyte analog:reporter conjugate indicates the presence of the analyte in the sample.

4. The device of claim 3, wherein a releasable analyte analog:reporter conjugate is bound to said molecular imprinted polymer,
wherein an affinity of the analyte for said binding sites of said molecular imprinted polymer is at least equal to an affinity of said analyte analog:reporter conjugate for said analyte-specific binding sites of said molecular imprinted polymer,
wherein upon contacting said molecular imprinted polymer with the analyte in the liquid sample, the analyte is bound and said analyte analog:reporter conjugate is displaced,
said device further comprising a results zone comprising an analyte analog:reporter conjugate binding element immobilized to said solid support on said flow path of the sample, said reporter conjugate binding element being capable of binding said displaced analyte analog:reporter conjugate,
wherein displacement of said analyte analog:reporter conjugate is proportional to a concentration of the analyte in the liquid sample, such that detecting binding of said analyte to said molecular imprinted polymer comprises detecting said binding of said displaced analyte:reporter conjugate to said analyte analog:reporter conjugate binding element.

5. The device of claim 1, wherein said analyte displaces a first binding agent:analyte conjugate releasably bound to said molecular imprinted polymer,
wherein said displaced first binding agent:analyte conjugate displaces a second:reporter conjugate releasably bound to said solid support,
wherein displacement of said second binding agent:reporter conjugate indicates the presence of the analyte in the sample.

6. The device of claim 5, wherein a releasable first binding agent:analyte conjugate is bound to said molecular imprinted polymer,
wherein an affinity of the analyte for said binding sites of said molecular imprinted polymer is at least equal to an affinity of said first binding agent:analyte conjugate for said analyte-specific binding sites of said molecular imprinted polymer,
wherein upon contacting said molecular imprinted polymer with the analyte in the liquid sample, the analyte is bound and said first binding agent:analyte conjugate is displaced,
said device further comprising:
i) a reporter-conjugate binding zone, comprising a reporter-conjugate binding element fixed to said solid support oh said flow path of the liquid sample, said reporter-conjugate binding element having binding sites with a detectable, releasable, second binding agent:reporter conjugate attached thereto,
wherein an affinity of said first binding agent:analyte conjugate for said analyte-specific binding sites of said reporter conjugate binding element is at least equal to an affinity of said second binding agent:reporter conjugate for said binding sites of said reporter:conjugate binding element, wherein binding of said first binding agent:analyte conjugate displaces second binding agent:reporter conjugate, and wherein displacement of said second binding agent:reporter conjugate is proportional to a concentration of the analyte in the liquid sample; and
ii) a results zone comprising a second binding agent:reporter conjugate binding element immobilized to said solid support on said flow path of the sample, said reporter conjugate binding element being capable of binding said displaced second binding agent:reporter conjugate,
wherein detecting binding of said analyte to said molecular imprinted polymer comprises detecting said binding of said second binding agent:reporter conjugate to said analyte analog:reporter conjugate binding element.

7. The device of claim 1, comprising an analyte analog:reporter conjugate, wherein said analyte competes with said analyte analog:reporter conjugate for said analyte-specific binding sites of said molecular imprinted polymer,
wherein the presence of unbound analyte analog:reporter conjugate is detected and indicates the presence of the analyte in the sample.

8. The device of claim 7, comprising an analyte analog:reporter conjugate, wherein an affinity of the analyte for said analyte-specific binding sites of said molecular imprinted polymer is at least equal to an affinity of said analyte analog:reporter conjugate for said analyte-specific binding sites of said molecular imprinted polymer,
wherein, upon contacting said molecular imprinted polymer with the analyte in the liquid sample, the analyte and said analyte analog:reporter conjugate compete for said binding sites of said molecular imprinted polymer
said device further comprising a results zone comprising an analyte analog:reporter conjugate binding element bound to said solid support, said reporter conjugate binding element being capable of binding said unbound analyte analog:reporter conjugate and providing a detectable signal that indicates the concentration of the analyte in the liquid sample.

9. The device of claim 1, comprising a first binding agent:analyte conjugate, a second binding agent:reporter conjugate, and a second binding agent:receptor conjugate binding element,
wherein said analyte competes with said first binding agent:analyte conjugate competes for said analyte-specific binding sites of said molecular imprinted polymer, such that in the presence of the analyte, at least a portion of said first binding agent:analyte conjugate is unbound,
wherein said unbound first binding agent:analyte conjugate competes with said second binding agent:reporter conjugate for binding to said second binding agent:receptor conjugate binding element.

10. The device of claim 9, comprising a first binding agent:analyte conjugate, wherein an affinity of the analyte for said analyte-specific binding sites of said molecular imprinted polymer is at least equal to an affinity of said first binding agent:analyte conjugate for said analyte-specific binding sites of said molecular imprinted polymer,
wherein upon contacting said molecular imprinted polymer with the analyte in the liquid sample, the analyte and said first binding agent:analyte conjugate analyte compete for said analyte-specific binding sites of said molecular imprinted polymer, and
wherein said unbound first binding agent:analyte conjugate flows in a flow path of the liquid sample,
said device further comprising:
i) a second binding agent:reporter application area, comprising a second binding agent:reporter conjugate in a dry state;
ii) a reporter conjugate binding zone downstream, comprising a reporter-conjugate binding element fixed to said solid support on said flow path of the liquid sample,
wherein an affinity of said first binding agent:analyte conjugate to said reporter-conjugate binding element is at least equal to an affinity of said second binding agent:reporter conjugate to said binding sites,
wherein upon contacting said dry second binding agent:reporter conjugate with said unbound first binding agent:analyte conjugate, said second binding agent:reporter conjugate and said unbound first binding agent:analyte conjugate compete for binding to said reporter-conjugate binding element,
wherein said unbound second binding agent:reporter conjugate flows downstream in said flow path of the liquid sample; and
iii) a results zone comprising a second binding agent:reporter conjugate binding element bound to said solid support, said reporter-conjugate binding element being capable of binding said unbound second binding agent:reporter conjugate and providing a detectable signal that indicates the concentration of the analyte in the liquid sample.

11. The device of claim 1, comprising a first binding agent:analyte analog, wherein unbound first binding agent:analyte analog is produced by at least one of competition with the analyte for binding sites of the molecular imprinted polymer and displacement by said analyte from said molecular imprinted polymer;
said device further comprising:
a second binding agent:reporter conjugate binding element, wherein an unbound second binding agent:reporter conjugate is produced by at least one of competition with said first binding agent:analyte conjugate and displacement by said first binding agent:analyte conjugate wherein the presence of unbound second binding agent:reporter conjugate indicates the presence of the analyte in the sample.

12. The device of claim 11, wherein said first binding agent:analyte analog is provided in said sample application area, or bound to said molecular imprinted polymer;
wherein said reporter conjugate binding element is fixed to said solid support on said flow path of the liquid sample, and said second binding agent:reporter conjugate is provided in a reporter conjugate application area or releasably bound to said reporter conjugate binding element,
said device further comprising a results zone comprising a second binding agent:reporter conjugate binding element bound to said solid support, said reporter-conjugate binding element being capable of binding said unbound second binding agent:reporter conjugate and providing a detectable signal that indicates the concentration of the analyte in the liquid sample.

13. The device of claim 4, wherein said reporter-conjugate-binding element is immobilized horizontally to the sample liquid flow path on said solid support,
wherein said unbound reporter conjugate saturates said binding sites of said reporter-conjugate binding element,
wherein, the results are visualized as an advancing column, having a length proportional to an amount of the analyte in the sample.

14. The device of claim 1, further comprising at least one of a positive control zone; a reference zone; an absorbent zone downstream of said results zone on said flow path of the liquid sample, said absorbent zone comprising absorbent material capable of absorbing the liquid sample; and a housing including at least one window aligned with at least one of the results zone, reference zone, and control zone to allow observation of test results on the device.

15. The device of claim 14, wherein the intensity of a signal in said results zone is compared with the intensity of a signal in said reference zone, the device being arranged and constructed to provide a positive result when the analyte is present at or above a threshold level, to indicate the concentration of the analyte in the sample.

16. The device of claim 14, wherein said reference zone comprises at least one discrete band of a binding element comprising a known quantity of said analyte analog:reporter conjugate, wherein the presence or absence of the target analyte in the sample is determined by comparison of the intensity of the signal from said reference zone with the intensity of said signal from said results zone.

17. The device of claim 14, wherein said device is devoid of a reference zone, wherein said results zone further comprises a scale parallel to the immobilized reporter-conjugate binding element calibrated to correspond to the analyte concentration in the liquid sample being analyzed and wherein the presence and concentration of analyte in the liquid is determined by the area covered by the reporter-conjugate along and through the reporter-conjugate binding element in the results zone.

18. The device of claim 1, wherein the analyte is selected from the group consisting of a cell, an organism, a small molecule, a protein, a hormone, an enzyme, a biomarker, metabolites of biomarkers, metabolites of drugs, a drug, a drug metabolite a drug-protein conjugate, a drug metabolite-protein conjugate, a vitamin, a drug of abuse, a natural or synthetic toxin, a chemical or biological warfare agent, antibodies to a drug, antibodies to infectious agents, an environmental pollutant, an immunoglobulin, a lymphokine, a cytokine, a soluble cancer antigen, a growth factor, a neurotransmitter, a molecule indicating the safety or quality of a foodstuff, a process chemical, a byproduct of a production process, a pesticide, an insecticide, a herbicide, a fertilizer, a surfactant, an adhesive, and an agent used in the manufacture of food, industrial agents or chemical products.

19. A kit comprising
a. the device of claim 1,
b. optionally, one or more reagents or compositions for extracting or processing the sample to elute the analyte;
c. optionally, one or more diluents; and
d. optionally, instructions for practicing a method of detecting and determining the presence, absence or concentration of a target analyte in a liquid sample.
